SEQUENCE LISTING (43 sequences)

SEQ ID NO: 1 - protein, 41 aa, Artificial Sequence
  Intracellular signalling domain
RSKRSRLLHSDYMNMTPRRPGPTRKHYQPYAPPRDFAAYRS

SEQ ID NO: 2 - protein, 242 aa, Artificial Sequence
  Anti-CD19 scFV domain
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 240 aa, Artificial Sequence
  scFV capable of binding to LMP2A peptide - MHC complex
QVQLKQSGPGLVQPSQSLSITCTVSGFSLTNYGVHWVRQSPGKGLEWLGVIWSGGSTDYNAAFISRLSISKDNSKSQVFFKMNSLQANDTAIYYCARNWVPYYFDYWGQGTTVTVSSGGGGSGGGGSGGGGSTDIVMTQSQKFMSTSVGDRVSVTCRASQNVFTNVAWYQQKPGQAPKALIYSTSYRYSGVPDRFTGSGSGTDFTLTISNVQSEDLAEYFCQQYISYPLTFGAGTKLELK

SEQ ID NO: 4 - protein, 21 aa, Artificial Sequence
  signal peptide
MALPVTALLLPLALLLHAARP

SEQ ID NO: 5 - DNA, 1563 bp, Artificial Sequence
  CD28-CAR
atggccttaccagtgaccgccttgctcctgccgctggccttgctgctccacgccgccaggccggacatccagatgacccagaccacaagctccctgtctgccagcctgggcgacagggtgacaatctcttgccgcgccagccaggacatctccaagtacctgaactggtatcagcagaagcctgacggcaccgtgaagctgctgatctaccacacatctcggctgcacagcggcgtgccatccaggttctccggctctggcagcggcaccgactattctctgacaatcagcaacctggagcaggaggacatcgccacctacttctgccagcagggcaataccctgccctatacatttggcggcggcaccaagctggagatcacaggaggaggaggcagcggcggaggaggctccggcggcggcggctctgaggtgaagctgcaggagtccggaccaggcctggtggcaccatcccagtctctgagcgtgacctgtacagtgagcggcgtgtccctgcctgactacggcgtgtcctggatcagacagccccctagaaagggcctggagtggctgggcgtgatctggggcagcgagacaacatactataattccgccctgaagtctcggctgaccatcatcaaggataactccaagtctcaggtgtttctgaagatgaatagcctgcagaccgacgatacagccatctactattgtgccaagcactactattacggggggagttatgctatggattattgggggcagggcaccagcgtcactgtctcatcacgggcggccgcacatcatcatcaccaccatcacggggccgcagaacaaaaactcatctcagaagaggatcgtacgaagcccaccacgacgccagcgccgcgaccaccaacaccggcgcccaccatcgcgtcgcagcccctgtccctgcgcccagaggcgtgccggccagcggcggggggcgcagtacacacgagggggctggacttcgcctgtgatacgcgtttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggatccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - protein, 520 aa, Artificial Sequence
  CD28 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRAAAHHHHHHHGAAEQKLISEEDRTKPTTTPAPRPPTPAPTIASQPLSLRPEACRPAAGGAVHTRGLDFACDTRFWVLVVVGGVLACYSLLVTVAFIIFWVRSKRSRLLHSDYMNMTPRRPGPTRKHYQPYAPPRDFAAYRSGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - DNA, 1689 bp, Artificial Sequence
  CD137/CD28-CAR
atggccttaccagtgaccgccttgctcctgccgctggccttgctgctccacgccgccaggccgcaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaagcacagactataatgcagctttcatatccagattgagcatcagcaaggacaattccaagagccaagttttctttaaaatgaacagtctgcaagctaatgacacagccatatattactgtgccagaaattgggtcccttactactttgactactggggccaaggcaccacggtcaccgtatcgagcggtggaggcggttcaggcggaggtggcagcggcggtggcgggtcgacggacattgtgatgacccagtctcaaaaattcatgtccacatcagttggagacagggtcagcgtcacctgcagggccagtcagaatgtgtttactaatgtagcctggtatcaacagaaaccagggcaagctcctaaagcactgatttactcgacatcctaccggtacagtggagtccctgatcgcttcacaggcagtggatctgggacagatttcactctcaccatcagcaatgtgcagtctgaagacttggcagagtatttctgtcagcaatatatcagctatcctctcacgttcggtgctgggaccaagctggagctgaaacgggcggccgcacatcatcatcaccaccatcacggggccgcagaacaaaaactcatctcagaagaggatcgtacgaagcccaccacgacgccagcgccgcgaccaccaacaccggcgcccaccatcgcgtcgcagcccctgtccctgcgcccagaggcgtgccggccagcggcggggggcgcagtacacacgagggggctggacttcgcctgtgatacgcgtttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccggatccaaacggggcagaaagaaactcctgtatatattcaaacaaccatttatgagaccagtacaaactactcaagaggaagatggctgtagctgccgatttccagaagaagaagaaggaggatgtgaactgggatccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 8 - protein, 562 aa, Artificial Sequence
  CD137/CD28-CAR
MALPVTALLLPLALLLHAARPQVQLKQSGPGLVQPSQSLSITCTVSGFSLTNYGVHWVRQSPGKGLEWLGVIWSGGSTDYNAAFISRLSISKDNSKSQVFFKMNSLQANDTAIYYCARNWVPYYFDYWGQGTTVTVSSGGGGSGGGGSGGGGSTDIVMTQSQKFMSTSVGDRVSVTCRASQNVFTNVAWYQQKPGQAPKALIYSTSYRYSGVPDRFTGSGSGTDFTLTISNVQSEDLAEYFCQQYISYPLTFGAGTKLELKRAAAHHHHHHHGAAEQKLISEEDRTKPTTTPAPRPPTPAPTIASQPLSLRPEACRPAAGGAVHTRGLDFACDTRFWVLVVVGGVLACYSLLVTVAFIIFWVRSKRSRLLHSDYMNMTPRRPGPTRKHYQPYAPPRDFAAYRSGSKRGRKKLLYIFKQPFMRPVQTTQEEDGCSCRFPEEEEGGCELGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - DNA, 20 bp, Artificial Sequence
  LCK gRNA1
gacccactggttacctacga

SEQ ID NO: 10 - DNA, 20 bp, Artificial Sequence
  LCK gRNA2
gccgggaaaagtgattcgag

SEQ ID NO: 11 - DNA, 20 bp, Artificial Sequence
  LCK gRNA3
cgggacttcgaccagaacca

SEQ ID NO: 12 - DNA, 20 bp, Artificial Sequence
  LCK gRNA4
tggagcccgaaccgtaagtg

SEQ ID NO: 13 - DNA, 20 bp, Artificial Sequence
  LCK gRNA5
cggcggattggagccttcgt

SEQ ID NO: 14 - DNA, 20 bp, Artificial Sequence
  LCK gRNA6
gcggaccagttcatgcaggc

SEQ ID NO: 15 - DNA, 20 bp, Artificial Sequence
  LCK gRNA7
cgttgtagtaccctgggtcg

SEQ ID NO: 16 - DNA, 20 bp, Artificial Sequence
  LCK gRNA8
gggtgacaatttccgtcagc

SEQ ID NO: 17 - DNA, 19 bp, Artificial Sequence
  LCK gRNA9
cgtaagtggggacccgtcg

SEQ ID NO: 18 - DNA, 20 bp, Artificial Sequence
  LCK gRNA10
ctggagcccgaaccgtaagt

SEQ ID NO: 19 - DNA, 20 bp, Artificial Sequence
  LCK gRNA11
ggtgaatgtcccgtagttaa

SEQ ID NO: 20 - DNA, 20 bp, Artificial Sequence
  LCK gRNA12
gatcgttttcactgtcggtc

SEQ ID NO: 21 - DNA, 20 bp, Artificial Sequence
  LCK gRNA13
ccgtaagtggggacccgtcg

SEQ ID NO: 22 - DNA, 20 bp, Artificial Sequence
  LCK gRNA14
ccctctcacgacggagatct

SEQ ID NO: 23 - DNA, 20 bp, Artificial Sequence
  LCK gRNA15
ctggcgcccgggaacactca

SEQ ID NO: 24 - DNA, 20 bp, Artificial Sequence
  LCK gRNA16
gtaagtggggacccgtcgtg

SEQ ID NO: 25 - DNA, 20 bp, Artificial Sequence
  LCK gRNA17
tgtgcccgttgtagtaccct

SEQ ID NO: 26 - DNA, 20 bp, Artificial Sequence
  LCK gRNA18
attacaccagtgagcccgac

SEQ ID NO: 27 - DNA, 20 bp, Artificial Sequence
  LCK gRNA19
tggcctagcacgcctcattg

SEQ ID NO: 28 - DNA, 20 bp, Artificial Sequence
  LCK gRNA20
cgtagagccgaaccagccgc

SEQ ID NO: 29 - DNA, 20 bp, Artificial Sequence
  LCK gRNA21
atccgtaatctggacaacgg

SEQ ID NO: 30 - DNA, 20 bp, Artificial Sequence
  LCK gRNA22
gccctctcacgacggagatc

SEQ ID NO: 31 - DNA, 20 bp, Artificial Sequence
  LCK gRNA23
atcgttttcactgtcggtcc

SEQ ID NO: 32 - DNA, 20 bp, Artificial Sequence
  LCK gRNA24
gaacctgagccgcaaggacg

SEQ ID NO: 33 - DNA, 20 bp, Artificial Sequence
  LCK gRNA25
ccattatcccatagtcccac

SEQ ID NO: 34 - DNA, 20 bp, Artificial Sequence
  LCK gRNA26
gctcacacccggaagatgac

SEQ ID NO: 35 - DNA, 20 bp, Artificial Sequence
  LCK gRNA27
ctcacggctccttcctcatc

SEQ ID NO: 36 - DNA, 20 bp, Artificial Sequence
  LCK gRNA28
aaggcgcagtccctgaccac

SEQ ID NO: 37 - DNA, 20 bp, Artificial Sequence
  LCK gRNA29
ctacgaaggctccaatccgc

SEQ ID NO: 38 - DNA, 20 bp, Artificial Sequence
  FYN gRNA1
agagttcacacctccaaaga

SEQ ID NO: 39 - DNA, 20 bp, Artificial Sequence
  FYN gRNA2
acggggaccttgcgtacgag

SEQ ID NO: 40 - DNA, 20 bp, Artificial Sequence
  FYN gRNA3
ttgtcctttggaaacccaag

SEQ ID NO: 41 - DNA, 20 bp, Artificial Sequence
  FYN gRNA4
gtcccccgaatcattccttg

SEQ ID NO: 42 - DNA, 20 bp, Artificial Sequence
  FYN gRNA5
tggatactacattaccaccc

SEQ ID NO: 43 - RNA, 100 nt, Artificial Sequence
  RNA sequence
  misc_feature (1)..(3): modified base with 2′-O-methyl 3′ phosphorothioate
  misc_feature (97)..(99): modified base with 2′-O-methyl 3′ phosphorothioate
gccgggaaaagugauucgagguuuuagagcuagaaauagcaaguuaaaauaaggcuaguccguuaucaacuugaaaaaguggcaccgagucggugcuuuu

(19) United States
(12) Patent Application Publication
(10) Pub. No.: US 2022/0160762 A1
(43) Pub. Date: May 26, 2022

(54) ENGINEERED IMMUNE CELLS

(71) Applicant: NATIONAL UNIVERSITY OF SINGAPORE, Singapore (SG)

(72) Inventors: Ling WU, Singapore (SG); Nicholas Robert John GASCOIGNE, Singapore (SG); Joanna BRZOSTEK, Singapore (SG)

(73) Assignee: NATIONAL UNIVERSITY OF SINGAPORE, Singapore (SG)

(21) Appl. No.: 17/434,186

(22) PCT Filed: Feb. 27, 2020
(86) PCT No.: PCT/SG2020/050090

§ 371 Date: Aug. 26, 2021

(30) Foreign Application Priority Data
Mar. 1, 2019 (SG) 10201901875X

(51) Int. Cl. A61K 35/17; C07K 16/08; A61P 35/00

(52) U.S. Cl. CPC A61K 35/17; A61P 35/00; C07K 16/085

(57) ABSTRACT

The present disclosure relates generally to the field of immunology. In particular, the disclosure relates to an immune cell expressing a CAR, wherein the immune cell has been modified such that the expression and/or function of LCK has been reduced or eliminated. The disclosure also relates to methods for treating a disease in a subject.

### FIELD

The present disclosure relates generally to the field of immunology. In particular, the disclosure relates to an immune cell expressing a chimeric antigen receptor (CAR) and methods for treating a disease in a subject.

### BACKGROUND

Immunotherapy has recently made unprecedented breakthroughs in treating cancer patients. In adoptive T-cell therapy, isolated human T cells are genetically-modified to enhance their specificity for a specific tumor antigen, such as by expression of a chimeric antigen receptor. Adoptive T-cell therapy involving chimeric antigen receptor T cells (CAR-T cells) is a major part of the immuno-oncology pipeline. To date, three generations of CAR-T technology have been developed and used in clinical trials for a number of cancers including B cell malignancies and multiple myeloma.

Despite its tremendous usefulness as a cancer treatment, adoptive immunotherapy with CAR-T cells has been limited, in part, by expression of endogenous T cell receptors on the cell surface. CAR-T cells expressing endogenous T cell receptors may recognize major and minor histocompatibility antigens following administration to an allogeneic patient. This has non-specific effects and can lead to the development of graft-versus-host-disease (GVHD) in patients.

Another limitation of T cell adoptive immunotherapy by CAR-T cells is that after reperfusion into the patient, the CAR-T cells start to show an “exhausted” phenotype due to the expression of inhibitory receptors such as PD-1, LAG-3, TIGIT and others, resulting in loss of T cell effector functions. This is a particular problem with solid tumours which often express the ligands for these inhibitory receptors (e.g. PD-L1 and PD-L2 for PD-1), and has limited the usefulness of CAR-T therapy for solid tumours.

Accordingly, there is a need to overcome, or at least to alleviate, one or more of the above-mentioned problems.

### SUMMARY OF THE INVENTION

Provided herein is an immune cell expressing a chimeric antigen receptor (CAR).

In one aspect, there is provided an immune cell expressing a chimeric antigen receptor (CAR), wherein the CAR comprises an intracellular signaling domain or fragment that is functional in the absence of lymphocyte-specific protein tyrosine kinase (LCK), and wherein the immune cell has been modified such that the expression and/or function of LCK has been reduced or eliminated.

In one embodiment, the intracellular signaling domain or fragment is functional in the presence of a dysfunctional LCK.

In one aspect, there is provided an immune cell expressing a CAR, wherein the immune cell has been modified such that expression or function of the LCK gene has been disrupted.

In one aspect, there is provided a method of manufacturing an immune cell as defined herein, the method comprising contacting an immune cell with an inhibitor of LCK for a sufficient time and under conditions to reduce or eliminate the expression and/or function of LCK.

In one aspect, there is provided a vector system comprising 1) a vector comprising a nucleic acid sequence encoding an inhibitor of LCK and 2) a vector comprising a nucleic acid sequence encoding a CAR.

In one aspect, there is provided a vector comprising a nucleic acid sequence encoding an inhibitor of LCK and a nucleic acid sequence encoding a CAR.

In one aspect, there is provided a method of improving the efficacy of a CAR-expressing immune cell in a cell therapy, the method comprising contacting the CAR-expressing immune cell with an inhibitor of LCK for a sufficient time and under conditions to reduce or eliminate the expression and/or function of LCK.

In one aspect, there is provided a method of treating a subject in need thereof, the method comprising administering an immune cell as defined herein for a sufficient time and under conditions to treat the subject.

In one aspect, there is provided an immune cell as defined herein for use in treating a subject in need thereof.

In one aspect, there is provided the use of an immune cell as defined herein in the manufacture of a medicament for treating a subject in need.

### BRIEF DESCRIPTION OF THE DRAWINGS

Some embodiments of the present invention will now be described by way of non-limiting example only, with reference to the accompanying drawings in which:

FIG. 1. Artificial antigen presenting CHO cell provides antigens for CAR and TCR T cell stimulation. (A) Schematic diagram of CAR construct. Myc-tag is used for detection of CAR expression. (B) Expression of LMP2A peptide (L2)-specific CAR or TCR after lentiviral transduction of Jurkat 76. CAR construct was stained using anti-Myc antibody, and TCR expression was identified by anti-CD3 antibody. (C) Endogenous TCR expression on three T cell lines used in this study. Jurkat 76 cell line was mostly used as the main cell line in the study. (D) Schematic diagram of single chain HLA construct with covalently fused peptide sequence as mono-peptide system is shown in the upper panel. The lower left panel shows the staining using L2-specific TCR-like Ab on each CHO-APC expressing specific antigen HLA-A2-L2 or irrelevant antigen HLA-A2-GAG. The lower right panel shows the responsiveness of L2-specific CAR-T to specific peptide or irrelevant peptide in mono-peptide system. Data are plotted as mean±SD of technical triplicates, from at least three experiments. (E) Schematic diagram of single chain HLA construct without covalently fused peptide sequence as multi-peptide system is shown in the upper panel. The lower left panel shows different peptides pulsed on the groove-open HLA-A2 and stained by corresponding specific TCR-like Ab. The lower right panel shows the responsiveness of EBNA1 peptide (E1), LMP1 peptide (L1), or LMP2A peptide (L2)-specific CAR-T to each specific peptide pulsed or unpulsed in multi-peptide system. Data are representative of at least three independent experiments and are plotted as mean±SD of technical triplicates. (*, p<0.05, and no significance (NS), P>0.05)

FIG. 2. Activation of TCR-like CAR is not enhanced by CD8 and transduces T cell signaling without LCK. (A) CD8 recruitment using total internal reflection microscopy (TIRFM). CD8α was labelled with mCherry fluorescent protein, and single chain HLA was covalently fused with Clover fluorescent protein. Left panel is representative of imaging data. Right panel is the quantified data of background corrected mean fluorescence intensity. Data are plotted as mean±SD, n>80 cell conjugates (****, p<0.0001), two independent experiments were performed. (B) Calcium flux of CAR-T and TCR-T with or without CD8. The curve is simulated by kinetic program of Flowjo. Data are representative of at least three independent experiments. (C) CAR and TCR responsiveness with or without CD8 coreceptor. Specific peptide L2 or irrelevant peptide epitope of GAG presented in mono-peptide system is labelled as CHO-L2 or CHO-GAG, respectively. Data are plotted as mean±SD of technical triplicates (***, p<0.001, and no significance (NS), p>0.05), from at least three independent experiments. (D) Western blot detection of LCK or FYN expression in CAR or TCR-Jcam1.6 cell (left panel). CAR or TCR responsiveness in LCK-deficient cell line Jcam1.6 (right panel). Data are representative of at least three independent experiments and are plotted as mean±SD of technical triplicates. (E) Calcium flux of CAR-Jcam cells. Curve is simulated by kinetic program of Flowjo. Data are representative of at least three independent experiments.

FIG. 3. LCK-independent CAR signaling requires CD28 costimulatory domain. (A) Schematic representation of CAR construct with the extracellular recognition domain of an anti-CD19 scFv. The responsiveness of this construct is shown on the right. (B) Schematic representation of the CAR construct with deletion of CD3ζ domain. The responsiveness of this construct is shown on the right. (C) Schematic representation of the CAR constructs with altered costimulatory domains: CD28 deletion (CAR-1), replacement with CD137 intracellular region (CD137-CAR) and the third generation CAR construct containing intracellular signaling domains from both CD28 and CD137 (CD28/CD137-CAR). The responsiveness of the constructs is shown on the right. (D) CD28 intracellular domain was mutated either by deletion or mutation at YMNM or PYAP motif. (E) Schematic representation of CD28 costimulation of CAR1 on JCam1.6 T cells by CHO-L2/CD80-CD86 cells, which express the specific antigen HLA-A2-L2 along with co-stimulators CD80 and CD86. The responsiveness of CAR1 or TCR co-stimulation with CD28 in Jcam1.6 cell is shown on the right. All data in this figure are representative of at least three independent experiments and are plotted as mean±SD of technical triplicates.

FIG. 4. CD28-CAR relies on FYN to transduce downstream signaling. (A) Responsiveness of CAR and TCR-Jurkat with SFK inhibitor PP2. Representative data of at least three experiments, plotted as mean±SD of technical triplicates. (B) Responsiveness of CAR and TCR-Jurkat in the presence of specific LCK or FYN inhibitor, A770041 or SU6656 respectively, at different concentrations (nM). The IL-2 production has been normalized to control as relative response (%) against log (inhibitor concentration), and data are plotted as mean±SD of technical triplicates. (C) Phosphorylation (pY420) of FYN at different timepoints. APC stands for specific CHO-L2. The number shown below indicates the relative band intensity of pY416 determined by calculating intensity value of pY420 band to that of total FYN. cCBL or PLCγ1 is shown as a loading control for CD28-CAR-Jcam or TCR-Jcam respectively. (D) Phosphorylation of TCR signaling pathway molecules PLCγ1, Erk, and CD3ζ at different timepoints by CAR, TCR and CAR-1. The relative band intensity of each phosphorylation was determined by calculating intensity value of pPLCγ1, pErk1/2, or pCD3ζ relative to that of total PLCγ1, Erk, or Erk, respectively. (E) LCK and FYN expression in LCK or FYN knock-out Jurkat after genetic editing by CRISPR-Cas9. (F) IL-2 production of TCR and CAR in LCK or FYN knock-out Jurkat. Data are representative of at least three independent experiments and are plotted as mean±SD of technical triplicates (*, p<0.05, and ****, p<0.0001).

FIG. 5. LCK-deficient CAR-T resets activation threshold and selectively allows only CAR triggering in T cells expressing both CAR and TCR. (A) CAR-Jurkat cells with different amounts of CAR expression responding to unpulsed HLA-A2 CHO APC or specific peptide-pulsed HLA-A2 CHO APC. Left and middle panels show CAR expression on CAR-Jurkat cells after sorting. Right panel shows the responsiveness of CAR-Jurkat with different amounts of CAR expression against specific peptide-pulsed or unpulsed CHO-HLA-A2. Data are plotted as mean±SD of technical triplicates, from three independent experiments. (B) Comparison of CAR activation specificity of CAR-Jcam, CAR-JE6-1, and CAR-Jurkat 76. Data are plotted as mean±SD of technical triplicates (*, p<0.05, **, p<0.01 and ****, p<0.0001), from three independent experiments. (C) Specificity of CAR-Jcam cells with or without exogeneous LCK. Left panel shows LCK expression after transduction, by western blot. FYN staining was used as control. Right panel shows the responsiveness of LCK-transduced or non-transduced CAR-Jcam cells towards unpulsed HLA-A2 CHO APC or specific peptide-pulsed HLA-A2 CHO APC. Data are plotted as mean±SD of technical triplicates (**, p<0.01 and ****, p<0.0001) from three independent experiments. (D) TCR and CAR responsiveness selectivity in LCK-sufficient or deficient Jurkat T cells. The upper panel shows the schematics of E183 peptide-specific TCR or LMP2 peptide-specific CARs expressed in LCK-sufficient or deficient Jurkat T cells. The lower panel shows the responsiveness of each group against CHO-E183 or CHO-L2 respectively. CHO-E183 is a mono-peptide CHO APC presenting only E183 peptide. Similarly, CHO-L2 presents only A2-LMP2 peptide. Data are representative of at least three independent experiments and are plotted as mean±SD of technical triplicates.

FIG. 6. LCK-deficient CAR-T cells express reduced PD-1 and show reduced CAR downregulation after stimulation. (A) PD-1 upregulation after stimulation of L2 specific CAR-T with CHO-L2 for 18 hrs. The amounts of CAR or TCR were detected by anti-Myc tag antibody or anti-CD3 antibody, respectively. The lower panels are the histograms of PD-1 and CAR or CD3 expression corresponding to the dot plot above. Data are representative of at least three independent experiments. (B) Schematic of serial stimulation experiment on CAR-T and TCR-T. The CAR-T and TCR-T were separated with specific CHO-APC and sampled for analysis at 18 hr, 42 hr, and 66 hr. After resting for 6 hrs, the CAR-T and TCR-T were restimulated at 24 hr and 48 hr. (C) PD-1 expression after serial stimulations. The PD-1 MFI was determined by measurements on the PD-1 positive population. Data are plotted as mean±SD of technical triplicates (****, p<0.0001). (D) CAR or TCR downregulation after serial stimulations. The percentage was determined by the ratio of total CAR or CD3 MFI after stimulation to that before stimulation. Data are plotted as mean±SD of technical triplicates (****, p<0.0001).

FIG. 7. CD8 recruitment and functional impact for CAR-T and TCR-T. (A) CD8α-mCherry transduction in CAR-T and TCR-T. (B) Synapse and CD8 recruitment in fluorescence microscope. Left panel is a representative image graph (n 5), GFP channel detected the presence of CHO-APCCD19, and mCherry detected the localization of CD8α-mCherry. The right panel are the quantified data of mean fluorescent intensity (MFI) ratio, which was calculated by mCherry MFI of synapse to that of outside synapse. Cut off value (1.5) is marked by a blue dashed line. Data are plotted as mean±SD, n≥5 cell conjugates (ns, p>0.05). (C) Co-transduction of CD8α and CD8β construct in CAR-Jurkat or TCR-Jurkat cells. CD8α and CD8β constructs were on different lentiviruses and co-transduced in CAR or TCR-Jurkat cells. (D) CAR and TCR expression after CD8α and CD8β co-transduction. The HLA-A2-L2 tetramer was used for CAR-T and TCR-T staining. TCR expression was also detected by anti-CD3 antibody. The total MFI is shown on the right side of each graph.

FIG. 8. Cell line CHO-APC-CD80/CD86 expression. (A) CD19 expression on Daudi or Jurkat cells. (B) CD28 expression on Jurkat or Jcam1.6 cell lines. (C) Co-expression of CD80 and CD86 on CHO-L2 APC. The CD80 and CD86 were linked by P2A cleavable linker. CD80-P2A-CD86 construct was on one lentivirus vector.

FIG. 9. Impact of SRC family kinases, LCK and FYN, on CAR-T and TCR-T. (A) IL-2 production of CAR-Jcam cell with or without SFK PP2. Data are plotted as mean±SD of technical triplicates. (Representative of ≥2 experiments) (B) IC50 of LCK- or FYN-specific inhibitors on CAR-Jurkat or TCR Jurkat cell. Left graph is with LCK inhibitor, right graph is with FYN inhibitor. (C) Calcium flux of CAR2-Jcam or CAR1-Jcam after specific HLA-A2-L2 tetramer was added into medium. The second generation CAR with CD28 costimulatory domain in Jcam1.6 cell is labelled as CAR2-Jcam, and the first generation of CAR in Jcam1.6 cell is labelled as CAR1-Jcam. (Representative of 3 experiments) (D) LCK KO and FYN KO single clone selection. Clone 20 in LCK KO was selected as Jurkat LCK KO cell, and Clone 8 in FYN KO was selected as Jurkat FYN KO cell. (E) The CAR or TCR expression detection on CAR-Jurkat FYN KO and CAR-Jurkat or on TCR-Jurkat FYN KO and TCR-Jurkat. CD3 was used as the indicator of TCR expression. (F) FYN and LCK expression in CAR-Jurkat FYN KO and CAR-Jurkat or on TCR-Jurkat FYN KO and TCR-Jurkat. c-CBL was used as internal control.

FIG. 10. Specificity and receptor selection of LCK-deficient CAR-T. (A) IL-2 production by CAR-Jcam with or without LCK. Representative data from at least three experiments, plotted as mean±SD of technical triplicates. (B) CAR or TCR expression on Jurkat. The TCR is specific for a peptide epitope from HBs antigen, E183. CAR was with the specificity as above, the peptide epitope (L2) from LMP2A protein.

FIG. 11. Systems for testing CAR signaling. (A) CD8 and CD4 expression of Jurkat 76, Jcam1.6, and JE6.1. (B) Electroporation of E1-CAR, L1-CAR and L2-CAR and their expression after 18 hrs. (n≥3). (C) HLA-A2 and specific peptide L2 presentation in the multi-peptide CHO-APC system. Peptide was pulsed for 3 hr at different concentrations in the medium. CHO-APC was then trypsinized and stained with either anti-HLA-A2 antibody (BB7.2) or L2 specific TCR-like antibody (n≥3).

FIG. 12. CAR or TCR expression after serial stimulation. CAR or TCR expression was detected by anti-myc or anti-CD3 antibody. (Representative of ≥3 experiments).

FIG. 13. Targeting the CAR to LCK locus turns primary CD8+ T cells into a more memory and less exhausted phenotype and results in enhanced in vivo efficacy. (A) Schematic of CRISPR/Cas9-targeted CAR integration into LCK locus. Upper, the LCK locus. Lower, DNA donor design containing CAR cassette with 1 kb flanking regions of left and right homologous arms. (B) Cytotoxicity assay of CD19-expressing Daudi and Raji cells as targets for LCK-locus CAR-T, conventional CAR-T, and control CD8⁺ T cells. The cytotoxicity (%) was calculated by the formula (LDH^{release}−negative)/(LDH^{release max}−negative). Data are representative of three independent experiments and plotted as mean±SD of technical triplicates (NS, p>0.05). (C) FACS analysis of CAR, exhaustion, and memory marker expression in T cells at resting state (Day 5 after sorting and restimulation by feeder cells). Representative of 2 donors. (D) Radar chart summary of exhaustion, and memory marker expression in T cells after encountering target cells at different E:T ratios. The axis is the percentage of expression in T cells. Data are representative of two independent experiments. (E) Schematic of in vivo animal model. The cancer cell administration was designated as day 0. (F) Kaplan-Meier analysis of survival of mice. N=5 mice per group, log-rank Mantel-Cox test. (G, H) Raji bearing mice were treated with 5×10⁶ CAR-T cells. Mice were euthanized on day 11 and day 18. Bone marrow CAR-T cells were analyzed, and the percentage of memory T cells (CD45RO⁺) was detected by FACS on day 11 and day 18. Exhaustion marker expression was analyzed on day 11. N=4 mice per group, dot=one mouse, CAR-T=blue square dot, LCK-locus CAR-T=red round dot. All data are means±SD (NS, p>0.05, *, p<0.05, and **, p<0.01).

FIG. 14. LCK locus CAR-targeted primary CAR-T cells. (A) percentage of CD8+ CAR+ CAR-T cells after LCK locus-targeted CRISPR/Cas9 editing. Representative data from at least three experiments. (B) LCK protein expression of sorted LCK-locus CAR-T cells. Left, comparison with conventional CAR-T. Right, the presence of truncated LCK in LCK-locus CAR-T cells. Representative data from at least three experiments. (C) Genotyping of the targeted site in the LCK gene. Forward primer: 5′-AGGGAGAGGTGGTGAAACATTA-3′, reverse primer: 5′-GAATGGAGTAGGGCATTGAAAG-3′. (D) Cytotoxicity of LCK-locus CAR-T to CD19⁺ Daudi cells and CD19⁻ Jurkat cells. Data are plotted as mean±SD of technical triplicates (****, p<0.0001, NS, p>0.05). (E) Exhaustion and memory marker expression on conventional CAR-T cells after encountering target cells at varying E:T ratios. Data are representative of two independent experiments. (F) Anti-human CD20 antibody staining in multi-organs from the survived mouse in 5×10⁶ LCK-locus CAR-T cells group. (G) CAR-T cells (CD8+/Live) and cancer (CD20⁺/Live) cells were detected and counted in bone marrow by FACS. N=4 mice per group (NS, p>0.05).

### DETAILED DESCRIPTION

In one aspect, there is provided an immune cell expressing a chimeric antigen receptor (CAR), wherein the CAR comprises an intracellular signaling domain or fragment that is functional in the absence of lymphocyte-specific protein tyrosine kinase (LCK), and wherein the immune cell has been modified such that the expression and/or function of LCK has been reduced or eliminated.

In one embodiment, there is provided an immune cell expressing a chimeric antigen receptor (CAR), wherein the CAR comprises an intracellular signaling domain or fragment that is functional in the absence of LCK or in the presence of dysfunctional LCK, and wherein the immune cell has been modified such that the expression and/or function of LCK has been reduced or eliminated.

Without being bound by theory, the inventors have found that the key signaling kinase in T cell receptor (TCR) signaling, the SRC-family kinase LCK, may be dispensable for CAR signaling. As LCK is crucial for TCR-mediated signaling, deleting or inhibiting LCK in a CAR-T cell may ensure that only antigen recognition by the CAR will lead to activation of the T cell. This may reduce off-target effects and therefore increase the safety of CAR technology. This may also help to avoid the occurrence of autoimmunity caused by endogenous TCRs and diminish the chance of graft versus host disease for allogeneic CAR-T cells.

The term “Chimeric Antigen Receptor” or “CAR” may refer to a set of polypeptides, which when in an immune effector cell, provides the cell with specificity for a target cell, typically a cancer cell, and with intracellular signal generation. A CAR may comprise at least an extracellular antigen binding domain, a transmembrane domain and a cytoplasmic signaling domain (also referred to herein as “an intracellular signaling domain”) comprising a functional signaling domain derived from a primary signaling domain and/or costimulatory domain as defined below. The set of polypeptides may be contiguous with each other. In some embodiments, the set of polypeptides include a dimerization switch that, upon the presence of a dimerization molecule, can couple the polypeptides to one another, e.g., can couple an antigen binding domain to an intracellular signaling domain.

In one embodiment, the CAR has a nucleic acid or amino acid sequence as shown in Table 1.

In one embodiment there is provided an immune cell expressing a chimeric antigen receptor (CAR), wherein the CAR comprises an intracellular signaling domain or fragment that is functional in the absence of LCK, and wherein the immune cell has been modified such that the expression and/or function of LCK has been knocked-out or knocked-down.

The term “intracellular signaling domain,” as used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In one embodiment there is provided an immune cell expressing a chimeric antigen receptor (CAR), wherein the CAR comprises CD28 or a fragment of CD28 that functional in the absence of LCK, and wherein the immune cell has been modified such that the expression and/or function of LCK has been knocked-out or knocked-down.

The intracellular signaling domain or fragment may be one that is functional in the absence of LCK or in the presence of a dysfunctional LCK. The intracellular signaling domain or fragment that is functional in the absence of LCK or in the presence of a dysfunctional LCK may be one that is able to interact with FYN and/or be phosphorylated by FYN and therefore trigger LCK-independent signaling. The intracellular signaling domain may, for example, comprise a signaling domain or fragment of ADD2, BCAR1, c-Raf, CBLC, CD28, CD36, CD44, CDH1, CHRNA7, CTNND1, CBL, CSF1R, DLG4, Dystroglycan, EPHA8, FYB, FASLG, GNB2L1, GRIN2A, ITK, Janus Kinase 2, KHDRBS1, LKB1, Nephrin, PAG1, PIK3R2, PRKCQ, PTK2B, PTK2, PTPRT, UNC119, RICS, SH2D1A, SKAP1, Syk, TNK2, TRPC6, Tau protein, TrkB, TYK2, TUBA3C, WAS or ZAP-70. The intracellular signaling domain or fragment that is functional in the absence of LCK or in the presence of a dysfunctional LCK may be CD28 or a fragment of CD28 that is able to trigger LCK-independent signaling. In one embodiment, the intracellular signaling domain comprises a signaling domain or fragment of a CD28 protein that is functional in the absence of LCK or in the presence of a dysfunctional LCK.

In one embodiment, the intracellular signaling domain comprises a signaling domain or fragment of a CD28 protein having a PYAP motif. In one embodiment, the intracellular signaling domain comprises the sequence of:

(SEQ ID NO: 1)
RSKRSRLLHSDYMNMTPRRPGPTRKHYQPYAPPRDFAAYRS.

In one embodiment, the intracellular signaling domain further comprises a primary-signaling domain comprising a functional signaling domain of a protein selected from CD3 zeta, CD3 gamma, CD3 delta, CD3 epsilon, common FcRgamma, Fc epsilon RI beta, CD79a, CD79b, Fcgamma RIIa, DAP10, or DAP12. The intracellular signaling domain may further comprise one or more functional signaling domains derived from at least one costimulatory domain as defined below. In one embodiment, the intracellular signaling domain comprises a costimulatory domain comprising a functional signaling domain of a protein selected from the group consisting of DAP10, CD28, CARD11, SLAMF1, LCK1, LCK3, LAT, OX40, CD27, CDS, ICAM-1, LFA-1 (CD11a/CD18), ICOS (CD278), and 4-1BB (CD137).

In one embodiment, the CAR comprises an extracellular antigen-binding domain. The antigen-binding domain may, for example, be an antibody or an antibody fragment. The antigen-binding domain can also be an autoantigen that can be recognized by autoantigen-specific B cell receptors on B lymphocytes, thus directing T cells to specifically target and kill autoreactive B lymphocytes in antibody-mediated autoimmune diseases. The antigen-binding domain may also be a peptide or protein ligand.

The term “antibody,” as used herein, may refer to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources.

The term “antibody fragment” may refer to at least one portion of an antibody that retains the ability to specifically interact with an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv.

In one embodiment, the “antibody fragment” is an scFV.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The CAR may comprise a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state.

Thus examples of cell surface markers that may act as ligands for the antigen moiety domain in the CAR include those associated with viral, bacterial and parasitic infections, autoimmune disease and cancer cells

The CAR can be engineered to target a tumor antigen of interest by way of engineering a desired antigen binding domain that specifically binds to an antigen on a tumor cell. In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those with skill in the art.

Tumor antigens are proteins that are produced by tumor cells that elicit an immune response, particularly T-cell mediated immune responses. The selection of the antigen binding domain of the invention will depend on the particular type of cancer to be treated. Tumor antigens are well known in the art and include, for example, a glioma-associated antigen, carcinoembryonic antigen (CEA), 3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, folate receptor (FRa) and mesothelin. In a preferred embodiment, the tumor antigen is selected from the group consisting of folate receptor (FRa), mesothelin, EGFRvIII, IL-13Ra, EGFR, CA-IX, MUC1, HER2, and any combination thereof. In one embodiment, the first CAR comprises an antigen binding domain which binds to mesothelin and the second CAR comprises an antigen binding domain that binds to FRa. In one embodiment, the CAR comprises an antigen binding domain that binds to HER2.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel17), tyrosinase, TRP-1, TRP-2 and tumor-specific multilineage antigens such as MAGI-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations: such as BCR-ABL, E2A-PRL, H4-RET, IGH-IGK, MYL-RAR; and viral antigens, such as the Epstein Barr virus antigens EBVA, LMP2 (e.g. LMP2A or LMP2B) and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In one embodiment, the scFV is an anti-CD19 scFV domain. The anti-CD19 scFV domain may, for example, have a sequence of:

(SEQ ID NO: 2)
a) DIQMTQTTSSLSASLGDRVTISCRASQDISKYLNWYQQKPDGTVKLL
IYHTSRLHSGVPSRFSGSGSGTDYSLTISNLEQEDIATYFCQQGNTLPYT
FGGGTKLEITGGGGSGGGGSGGGGSEVKLQESGPGLVAPSQSLSVTCTVS
GVSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSS.

In one embodiment, the scFV is an scFVC capable of binding to a peptide-MHC complex presenting one of the LMP2A protein peptides. The scFV may, for example, have a sequence of:

(SEQ ID NO: 3)
a) QVQLKQSGPGLVQPSQSLSITCTVSGFSLTNYGVHWVRQSPGKGLEW
LGVIWSGGSTDYNAAFISRLSISKDNSKSQVFFKMNSLQANDTAIYYCAR
NWVPYYFDYWGQGTTVTVSSGGGGSGGGGSGGGGSTDIVMTQSQKFMSTS
VGDRVSVTCRASQNVFTNVAWYQQKPGQAPKALIYSTSYRYSGVPDRFTG
SGSGTDFTLTISNVQSEDLAEYFCQQYISYPLTFGAGTKLELK.

In one embodiment, the scFV is an anti-LMP2 scFV domain.

The “antibody fragment” may comprise of other antibody of antibody fragment sequences that are known in the art, depending on the antigen that is to be targeted.

In one embodiment, the chimeric antigen receptor further comprises a signal peptide. The signal peptide may, for example, have a sequence of MALPVTALLLPLALLLHAARP (SEQ ID NO: 4).

The CAR may be a CAR having a protein sequence as shown in Table 1.

In one embodiment, the immune cell is a recombinant immune cell. The term “recombinant” includes reference to a cell that has been modified by the introduction of a heterologous nucleic acid, or a cell derived from a cell that has been modified in such a manner, but does not encompass the alteration of the cell by naturally occurring events (e.g., spontaneous mutation, natural transformation, natural transduction, natural transposition) such as those occurring without deliberate human intervention. The recombinant immune cell may be a non-naturally occurring cell. The recombinant immune cell may also be an engineered cell. In one embodiment, the recombinant immune cell is an engineered immune cell such as an engineered T-cell or engineered NK cell. In one embodiment, the recombinant immune cell is an isolated immune cell.

In one embodiment, the immune cell is a T cell or an NK cell.

The term “immune cell” as referred to herein includes cells that are of haematopoietic origin and that play a role in the immune response. Immune cells include lymphocytes, such as B cells and T cells; natural killer (NK) cells; myeloid cells, such as monocytes, macrophages, dendritic cells, eosinophils, mast cells, basophils, and granulocytes.

In one embodiment, the immune cell is an immune effector cell. The term “immune effector cell,” as that term is used herein, refers to a cell that is involved in an immune response, e.g., in the promotion of an immune effector response. Examples of immune effector cells include T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation of a target cell. In the case of a T cell, primary stimulation and co-stimulation are examples of immune effector function or response.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

As used herein, the term “T cell” includes CD4+ T cells and CD8+ T cells. The term T cell also includes both T helper 1 type T cells and T helper 2 type T cells and also Th-IL 17 cells.

In one embodiment, the immune cell has been modified such that the expression and/or function of LCK has been reduced or eliminated. The immune cell may, for example, be modified to obtain an LCK knock-out or knock down. The term “knock-out” may refer to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. On the other hand, the term “knock-down” may refer to reduction in the expression of a gene or its gene product(s). As a result of a gene knock-down, the protein activity or function may be attenuated or the protein levels may be reduced or eliminated.

In one embodiment, the immune cell comprises or has been contacted with an inhibitor of LCK.

The inhibitor of LCK may be a nucleic acid sequence capable of downregulating or eliminating gene expression or modifying the function of LCK.

The nucleic acid may be a nucleic acid that is capable of downregulating or eliminating gene expression or modifying the function of LCK is selected from the group consisting of an antisense RNA, antagomir RNA, siRNA, shRNA, CRISPR system, a zinc finger nuclease system and a transcription activator-like effector based nuclease (TALEN) system. In one embodiment, the nucleic acid encodes an intracellular antibody that is coupled with a protease that degrades LCK or indirectly leads to the degradation of LCK in the cell.

It is known in the art that it is possible to use a site-specific nuclease to make a DNA break in the genome of a living cell, and that such a DNA break can result in permanent modification of the genome via mutagenic Non-homologous end joining (NHEJ) repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. The use of nucleases to induce a double-strand break in a target locus is known to stimulate homologous direct repair (HDR), particularly of transgenic DNA sequences flanked by sequences that are homologous to the genomic target. In this manner, exogenous nucleic acid sequences can be inserted into a target locus. Such exogenous nucleic acids can encode, for example, a chimeric antigen receptor, an exogenous TCR, or any sequence or polypeptide of interest.

In different embodiments, a variety of different types of nuclease are useful for practicing the invention. In one embodiment, the invention can be practiced using recombinant meganucleases. In another embodiment, the invention can be practiced using a CRISPR nuclease. Methods for making CRISPRs that recognize pre-determined DNA sites are known in the art. In another embodiment, the invention can be practiced using TALENs or Compact TALENs. In a further embodiment, the invention can be practiced using megaTALs.

Engineered endonucleases based on the CRISPR/Cas9 system are also known in the art. A CRISPR endonuclease comprises two components: (1) a caspase effector nuclease, typically microbial Cas9; and (2) a short “guide RNA” comprising a nucleotide targeting sequence that directs the nuclease to a location of interest in the genome.

The term “CRISPR” refers to a caspase-based endonuclease comprising a caspase, such as Cas9, and a guide RNA that directs DNA cleavage of the caspase by hybridizing to a recognition site in the genomic DNA. A guide RNA for knock-out may be one that is shown in Table 2. In one embodiment, a guide RNA is used with SEQ ID NO: 43 to knock-down the expression of the LCK gene in a cell. In one embodiment, the guide RNA is SEQ ID NO: 10.

In one embodiment, the inhibitor of LCK is an inhibitor of LCK protein. The inhibitor of LCK may be an inhibitor of LCK kinase activity. The inhibitor of LCK may be selected from the group consisting of Aminoquinazolin, A-420983, A770041, Dasatinib, Saractinib and Masatinib.

In one embodiment, the immune cell has reduced PD-1 expression as compared to a cell that has not been modified such that the expression and/or function of LCK has been reduced or eliminated. This may reduce the tendency towards T cell exhaustion. This may also improve CAR-T cell responses against solid tumours.

In one embodiment, the immune cell comprises a vector comprising a nucleic acid encoding the CAR. In one embodiment, the immune cell comprises a vector comprising a nucleic acid encoding an inhibitor of LCK capable of downregulating or eliminating gene expression of LCK. In one embodiment, the immune cell comprises a vector comprising a nucleic acid encoding the CAR and a nucleic acid encoding an inhibitor of LCK capable of downregulating or eliminating gene expression of LCK.

The term “vector” or “expression construct” may refer to a nucleic acid molecule containing a desired coding sequence and appropriate nucleic acid sequences necessary for expression of the operably linked coding sequence (e.g. an insert sequence that codes for a product) in a particular cell. An expression vector construct may comprise sufficient cis-acting elements for expression; other elements for expression can be supplied by the host cell or in an in vitro expression system. Expression vector constructs include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The “vector” may also be a “transfer vector” which refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some embodiments, the CAR sequences are delivered into cells using a retroviral or lentiviral vector. CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

The term “expression” may refer to the transcription and/or translation of a particular nucleotide sequence driven by a promoter.

The term “encode” or “encoding” includes reference to nucleotides and/or amino acids that correspond to other nucleotides or amino acids in the transcriptional and/or translational sense.

The term “nucleic acid” includes a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The terms “nucleic acid”, “nucleic acid molecule”, “nucleic acid sequence” and “polynucleotide” are used interchangeably herein unless the context indicates otherwise. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, siRNAs, shRNAs, RNAi agents, and primers. A polynucleotide can be modified or substituted at one or more base, sugar and/or phosphate, with any of various modifications or substitutions described herein or known in the art. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form. In some contexts, the terms “nucleic acid” or “polynucleotide” and the like encompass any material which conveys genetic information or performs a function of a nucleic acid or polynucleotide (e.g., it can be translated into a protein or act as an RNAi agent), even if such material is not strictly composed of nucleotides (which consist of a sugar, base and phosphate); such genetic material may comprise, as non-limiting examples, peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), and/or unlocked nucleic acid (UNA).

The terms “protein” and “polypeptide” are used interchangeably and may refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

Provided herein is an immune cell expressing a CAR, wherein the immune cell has been modified such that the LCK gene has been disrupted. Provided herein is an immune cell expressing a CAR, wherein the immune cell has been modified such that the expression or the function of the LCK gene has been disrupted. In one embodiment, there is provided a CART cell wherein the LCK gene has been disrupted. In one embodiment, there is provided a CART cell wherein the expression or the function of the LCK gene has been disrupted. In one embodiment, there is provided a method of disrupting the LCK gene in a CART cell or an immune cell expressing a CAR. In one embodiment, there is provided a method of disrupting the expression or function of the LCK gene in a CART cell or an immune cell expressing a CAR.

The terms “disruption” and “disrupted” are used interchangeably herein to refer to any genetic modification that decreases or eliminates expression and/or the functional activity of the nucleic acid or an expression product thereof. For example, disruption of a gene includes within its scope any genetic modification that decreases or eliminates expression of the gene and/or the functional activity of a corresponding gene product (e.g., mRNA and/or protein). Genetic modifications include complete or partial inactivation, suppression, deletion, interruption, blockage, or downregulation of a nucleic acid (e.g., a gene). Illustrative genetic modifications include, but are not limited to, gene knockout, inactivation, mutation (e.g., insertion, deletion, point, or frameshift mutations that disrupt the expression or activity of the gene product), or use of inhibitory nucleic acids (e.g., inhibitory RNAs such as sense or antisense RNAs, molecules that mediate RNA interference such as siRNA, shRNA, miRNA; etc.), inhibitory polypeptides (e.g., antibodies, polypeptide-binding partners, dominant negative polypeptides, enzymes etc.) or any other molecule that inhibits the activity of the LCK gene or level or functional activity of an expression product of the LCK gene.

In one aspect, there is provided a method of manufacturing (or preparing) an immune cell as defined herein, the method comprising contacting an immune cell with an inhibitor of LCK for a sufficient time and under conditions to reduce or eliminate the expression and/or function of LCK. The inhibitor of LCK may be a nucleic acid sequence capable of downregulating or eliminating gene expression or modifying the function of LCK. In one embodiment, the inhibitor of LCK is a CRISPR system. The method may further comprise introducing a nucleic acid encoding a CAR into the immune cell. The method may comprise a prior step of obtaining an immune cell from the patient.

In one embodiment, there is provided a nucleic acid encoding an inhibitor of LCK. In one embodiment, there is provided a nucleic acid encoding a CAR. In one embodiment, there is provided a nucleic acid comprising a nucleic acid encoding an inhibitor of LCK and a nucleic acid encoding a CAR.

In one aspect, there is provided a vector system comprising 1) a vector comprising a nucleic acid sequence encoding an inhibitor of LCK and 2) a vector comprising a nucleic acid sequence encoding a CAR.

In one aspect, there is provided a vector comprising a nucleic acid sequence encoding an inhibitor of LCK and a nucleic acid sequence encoding a CAR.

In one embodiment, the nucleic acid sequence encoding a CAR is also an inhibitor of LCK.

In one embodiment, the invention provides a pharmaceutical composition comprising an immune cell or a vector as described herein, and a pharmaceutically acceptable carrier.

By “pharmaceutically acceptable carrier” is meant a solid or liquid filler, diluent or encapsulating substance that can be safely used in topical or systemic administration to an animal, preferably a mammal, including humans. Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

In one aspect, there is provided a method of improving the efficacy of a CAR-expressing immune cell in a cell therapy, the method comprising contacting the CAR-expressing immune cell with an inhibitor of LCK for a sufficient time and under conditions to reduce or eliminate the expression and/or function of LCK.

In one embodiment, the CAR-expressing immune cell is a CART cell.

In one embodiment, the off-target effects of the CAR-expressing immune cell are reduced. In one embodiment, the exhaustion phenotype in the CAR-expressing cell is reduced. In one embodiment, the memory of the CAR-expressing immune cell is improved.

Disclosed herein is a method of reducing off-target effects of a CAR-expressing immune cell in a cell therapy, the method comprising contacting the CAR-expressing immune cell with an inhibitor of LCK for a sufficient time and under conditions to reduce or eliminate the expression and/or function of LCK.

Disclosed herein is a method of reducing the exhaustion phenotype of a CAR-expressing immune cell in a cell therapy, the method comprising contacting the CAR-expressing immune cell with an inhibitor of LCK for a sufficient time and under conditions to reduce or eliminate the expression and/or function of LCK.

Disclosed herein is a method of improving the memory of a CAR-expressing immune cell in a cell therapy, the method comprising contacting the CAR-expressing immune cell with an inhibitor of LCK for a sufficient time and under conditions to reduce or eliminate the expression and/or function of LCK.

In one aspect, there is provided a method of treating a subject in need thereof, the method comprising administering an immune cell as defined herein for a sufficient time and under conditions to treat the subject.

In one embodiment, the subject has a disease associated with expression of a tumor antigen (e.g., a proliferative disease, a precancerous condition, a cancer, and a non-cancer related indication associated with expression of the tumor antigen).

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In one aspect, a cancer associated with expression of a tumor antigen as described herein is a hematological cancer. In one aspect, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein. In an embodiment, the tumor antigen-expressing cells overexpresses the tumor antigen protein.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a haematological malignancy characterized by overexpression of the tumor antigen can be determined by standard assays known in the art.

In one embodiment, the subject has an infectious disease. The infectious disease may lead to expression of one or more infection markers (such as bacterial or viral markers) on the surface of infected cells which may be targeted by an immune cell of the present invention. The infection may, for example, be an infection by Epstein-Bar Virus.

In one embodiment, the subject has an autoimmune disease such as rheumatoid arthritis, psoriasis or systemic lupus erythematosus.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a sub-stage of a Stage II cancer. One skilled in the art will appreciate that the classification of a Stage II cancer as either an early stage cancer or a late stage cancer depends on the particular type of cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), Burkitt's lymphoma, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In one embodiment, there is provided a method of immunizing a subject from a disease, the method comprising administering an immune cell as defined herein for a sufficient time and under conditions to immunize the subject.

The term “administering” refers to contacting, applying, injecting, transfusing or providing a composition of the present invention to a subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender.

The methods as defined herein may comprise administering an effective amount of an immune cell to a subject in need. The term “effective amount” as defined herein is meant the administration of an amount of agent to an individual in need thereof, either in a single dose or as part of a series, that is effective for that elicitation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials

In one aspect, there is provided an immune cell as defined herein for use in treating a subject in need thereof.

In one aspect, there is provided the use of an immune cell as defined herein in the manufacture of a medicament for treating a subject in need.

The immune cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Ex vivo procedures are well known in the art and are discussed more fully below. Briefly, cells are isolated from a mammal (e.g. a human) and genetically modified (i.e. transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient. In addition to using a cell-based vaccine in terms of ex vivo immunization, also included in the methods described herein are compositions and methods for in vivo immunization to elicit an immune response directed against an antigen in a patient.

Provided herein is the use of an immune cell as defined herein.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Certain embodiments of the invention will now be described with reference to the following examples which are intended for the purpose of illustration only and are not intended to limit the scope of the generality herein before described.

### EXAMPLES

### Materials and Methods

Plasmids and Sequences

The lentiviral vector and its associated packaging plasmids were purchased from Vectorbuilder. Chimeric antigen receptor with CD28 and CD137 costimulatory sequences was synthesized and cloned into lentiviral vector by Vectorbuilder. Human CD8A, CD8B, CD80, CD86 and LCK genes were cloned from a human cDNA library in-house. The scFv constructs for TCR-like antibodies were produced in Paul A. Macary's lab (NUS), and TCRs specific for HLA-A*02:01 with peptide LMP2A₄₂₆₋₄₃₄ (from EBV) and A*02:01 with peptide E183-91 (from HBV) were generous gifts from Hans Stauss (University College London) and Antonio Bertoletti (Duke-NUS Medical School), respectively. Single-chain trimer GAG-HLA-A2 was a gift from Keith Gould (Imperial College London). Peptide mutagenesis: GAG (SLYNTVATL) to LMP2A₄₂₆₋₄₃₄ (CLGGLLTMV) (L2), LMP1₁₂₅₋₁₃₃ (YLLEMLWRL) (L1), EBNA1₅₆₂₋₅₇₀ (FMVFLQTHI) (E1), E183-91 (FLLTRILTI) (E183) or deletion in Single-chain trimer GAG-HLA-A2; CD28 Y170F, P187,190A, and intracellular domain deletion mutations were all done by using Q5 mutagenesis Kit (New England Biolabs). All the molecular cloning work was done by using In-Fusion HD cloning kit (Clontech), and Single-chain trimer MHC constructs were cloned into pcDNA3-Clover (Addgene plasmid #40259) for generating artificial antigen presenting CHO cell.

TABLE 1
DNA and amino acid sequences of the second generation CAR and third generation
CAR which have both shown LCK-independent signaling. The highlighted portion
in the CD28-CAR sequences shows the antigen-binding domain (which is an
anti-CD19 scFV domain). The antigen-binding domain may be substituted with
other antigen-binding domains (such as other scFVs or antibodies) that
can provide a different antigen specificity.

| CD28-CAR (DNA sequence) | CD28-CAR (Protein sequence) |
|---|---|
| ATGGCCTTACCAGTGACCGCCTTGCTCCTGC | MALPVTALLLPLALLLHAARPDIQMTQTTSSLS |
| CGCTGGCCTTGCTGCTCCACGCCGCCAGGCC | ASLGDRVTISCRASQDISKYLNWYQQKPDGTV |
| GGACATCCAGATGACCCAGACCACAAGCTCC | KLLIYHTSRLHSGVPSRFSGSGSGTDYSLTISNL |
| CTGTCTGCCAGCCTGGGCGACAGGGTGACAA | EQEDIATYFCQQGNTLPYTFGGGTKLEITGGG |
| TCTCTTGCCGCGCCAGCCAGGACATCTCCAA | GSGGGGSGGGGSEVKLQESGPGLVAPSQSLSV |
| GTACCTGAACTGGTATCAGCAGAAGCCTGAC | TCTVSGVSLPDYGVSWIRQPPRKGLEWLGVIW |
| GGCACCGTGAAGCTGCTGATCTACCACACAT | GSETTYYNSALKSRLTIIKDNSKSQVFLKMNSL |
| CTCGGCTGCACAGCGGCGTGCCATCCAGGTT | QTDDTAIYYCAKHYYYGGSYAMDYWGQGTS |
| CTCCGGCTCTGGCAGCGGCACCGACTATTCT | VTVSSRAAAHHHHHHHGAAEQKLISEEDRTKP |
| CTGACAATCAGCAACCTGGAGCAGGAGGAC | TTTPAPRPPTPAPTIASQPLSLRPEACRPAAGGA |
| ATCGCCACCTACTTCTGCCAGCAGGGCAATA | VHTRGLDFACDTRFWVLVVVGGVLACYSLLV |
| CCCTGCCCTATACATTTGGCGGCGGCACCAA | TVAFIIFWVRSKRSRLLHSDYMNMTPRRPGPT |
| GCTGGAGATCACAGGAGGAGGAGGCAGCGG | RKHYQPYAPPRDFAAYRSGSRVKFSRSADAPA |
| CGGAGGAGGCTCCGGCGGCGGCGGCTCTGA | YQQGQNQLYNELNLGRREEYDVLDKRRGRDP |
| GGTGAAGCTGCAGGAGTCCGGACCAGGCCT | EMGGKPQRRKNPQEGLYNELQKDKMAEAYSE |
| GGTGGCACCATCCCAGTCTCTGAGCGTGACC | IGMKGERRRGKGHDGLYQGLSTATKDTYDAL |
| TGTACAGTGAGCGGCGTGTCCCTGCCTGACT | HMQALPPR (SEQ ID NO: 6) |
| ACGGCGTGTCCTGGATCAGACAGCCCCCTAG | |
| AAAGGGCCTGGAGTGGCTGGGCGTGATCTG | |
| GGGCAGCGAGACAACATACTATAATTCCGCC | |
| CTGAAGTCTCGGCTGACCATCATCAAGGATA | |
| ACTCCAAGTCTCAGGTGTTTCTGAAGATGAA | |
| TAGCCTGCAGACCGACGATACAGCCATCTAC | |
| TATTGTGCCAAGCACTACTATTACGGGGGGA | |
| GTTATGCTATGGATTATTGGGGGCAGGGCAC | |
| CAGCGTCACTGTCTCATCACGGGCGGCCGCA | |
| CATCATCATCACCACCATCACGGGGCCGCAG | |
| AACAAAAACTCATCTCAGAAGAGGATCGTA | |
| CGAAGCCCACCACGACGCCAGCGCCGCGAC | |
| CACCAACACCGGCGCCCACCATCGCGTCGCA | |
| GCCCCTGTCCCTGCGCCCAGAGGCGTGCCGG | |
| CCAGCGGCGGGGGGCGCAGTACACACGAGG | |
| GGGCTGGACTTCGCCTGTGATACGCGTTTTT | |
| GGGTGCTGGTGGTGGTTGGTGGAGTCCTGGC | |
| TTGCTATAGCTTGCTAGTAACAGTGGCCTTT | |
| ATTATTTTCTGGGTGAGGAGTAAGAGGAGCA | |
| GGCTCCTGCACAGTGACTACATGAACATGAC | |
| TCCCCGCCGCCCCGGGCCCACCCGCAAGCAT | |
| TACCAGCCCTATGCCCCACCACGCGACTTCG | |
| CAGCCTATCGCTCCGGATCCAGAGTGAAGTT | |
| CAGCAGGAGCGCAGACGCCCCCGCGTACCA | |
| GCAGGGCCAGAACCAGCTCTATAACGAGCTC | |
| AATCTAGGACGAAGAGAGGAGTACGATGTT | |
| TTGGACAAGAGACGTGGCCGGGACCCTGAG | |
| ATGGGGGGAAAGCCGCAGAGAAGGAAGAAC | |
| CCTCAGGAAGGCCTGTACAATGAACTGCAGA | |
| AAGATAAGATGGCGGAGGCCTACAGTGAGA | |
| TTGGGATGAAAGGCGAGCGCCGGAGGGGCA | |
| AGGGGCACGATGGCCTTTACCAGGGTCTCAG | |
| TACAGCCACCAAGGACACCTACGACGCCCTT | |
| CACATGCAGGCCCTGCCCCCTCGCTAA | |
| (SEQ ID NO: 5) | |
| CD137/CD28-CAR (DNA sequence) | CD137/CD28-CAR (Protein sequence) |
| ATGGCCTTACCAGTGACCGCCTTGCTCCT | MALPVTALLLPLALLLHAARPQVQLKQSGPGL |
| GCCGCTGGCCTTGCTGCTCCACGCCGCC | VQPSQSLSITCTVSGFSLTNYGVHWVRQSPGK |
| AGGCCGCAGGTGCAGCTGAAGCAGTCA | GLEWLGVIWSGGSTDYNAAFISRLSISKDNSKS |
| GGACCTGGCCTAGTGCAGCCCTCACAGA | QVFFKMNSLQANDTAIYYCARNWVPYYFDY |
| GCCTGTCCATCACCTGCACAGTCTCTGGT | WGQGTTVTVSSGGGGSGGGGSGGGGSTDIVM |
| TTCTCATTAACTAACTATGGTGTACACTG | TQSQKFMSTSVGDRVSVTCRASQNVFTNVAW |
| GGTTCGCCAGTCTCCAGGAAAGGGTCTG | YQQKPGQAPKALIYSTSYRYSGVPDRFTGSGS |
| GAGTGGCTGGGAGTGATATGGAGTGGTG | GTDFTLTISNVQSEDLAEYFCQQYISYPLTFGA |
| GAAGCACAGACTATAATGCAGCTTTCAT | GTKLELKRAAAHHHHHHHGAAEQKLISEEDR |
| ATCCAGATTGAGCATCAGCAAGGACAAT | TKPTTTPAPRPPTPAPTIASQPLSLRPEACRPAA |
| TCCAAGAGCCAAGTTTTCTTTAAAATGA | GGAVHTRGLDFACDTRFWVLVVVGGVLACYS |
| ACAGTCTGCAAGCTAATGACACAGCCAT | LLVTVAFIIFWVRSKRSRLLHSDYMNMTPRRP |
| ATATTACTGTGCCAGAAATTGGGTCCCT | GPTRKHYQPYAPPRDFAAYRSGSKRGRKKLLY |
| TACTACTTTGACTACTGGGGCCAAGGCA | IFKQPFMRPVQTTQEEDGCSCRFPEEEEGGCEL |
| CCACGGTCACCGTATCGAGCGGTGGAGG | GSRVKFSRSADAPAYQQGQNQLYNELNLGRR |
| CGGTTCAGGCGGAGGTGGCAGCGGCGGT | EEYDVLDKRRGRDPEMGGKPQRRKNPQEGLY |
| GGCGGGTCGACGGACATTGTGATGACCC | NELQKDKMAEAYSEIGMKGERRRGKGHDGLY |
| AGTCTCAAAAATTCATGTCCACATCAGT | QGLSTATKDTYDALHMQALPPR |
| TGGAGACAGGGTCAGCGTCACCTGCAGG | (SEQ ID NO: 8) |
| GCCAGTCAGAATGTGTTTACTAATGTAG | |
| CCTGGTATCAACAGAAACCAGGGCAAGC | |
| TCCTAAAGCACTGATTTACTCGACATCCT | |
| ACCGGTACAGTGGAGTCCCTGATCGCTT | |
| CACAGGCAGTGGATCTGGGACAGATTTC | |
| ACTCTCACCATCAGCAATGTGCAGTCTG | |
| AAGACTTGGCAGAGTATTTCTGTCAGCA | |
| ATATATCAGCTATCCTCTCACGTTCGGTG | |
| CTGGGACCAAGCTGGAGCTGAAACGGG | |
| CGGCCGCACATCATCATCACCACCATCA | |
| CGGGGCCGCAGAACAAAAACTCATCTCA | |
| GAAGAGGATCGTACGAAGCCCACCACG | |
| ACGCCAGCGCCGCGACCACCAACACCGG | |
| CGCCCACCATCGCGTCGCAGCCCCTGTC | |
| CCTGCGCCCAGAGGCGTGCCGGCCAGCG | |
| GCGGGGGGCGCAGTACACACGAGGGGG | |
| CTGGACTTCGCCTGTGATACGCGTTTTTG | |
| GGTGCTGGTGGTGGTTGGTGGAGTCCTG | |
| GCTTGCTATAGCTTGCTAGTAACAGTGG | |
| CCTTTATTATTTTCTGGGTGAGGAGTAAG | |
| AGGAGCAGGCTCCTGCACAGTGACTACA | |
| TGAACATGACTCCCCGCCGCCCCGGGCC | |
| CACCCGCAAGCATTACCAGCCCTATGCC | |
| CCACCACGCGACTTCGCAGCCTATCGCT | |
| CCGGATCCAAACGGGGCAGAAAGAAAC | |
| TCCTGTATATATTCAAACAACCATTTATG | |
| AGACCAGTACAAACTACTCAAGAGGAA | |
| GATGGCTGTAGCTGCCGATTTCCAGAAG | |
| AAGAAGAAGGAGGATGTGAACTGGGAT | |
| CCAGAGTGAAGTTCAGCAGGAGCGCAG | |
| ACGCCCCCGCGTACCAGCAGGGCCAGAA | |
| CCAGCTCTATAACGAGCTCAATCTAGGA | |
| CGAAGAGAGGAGTACGATGTTTTGGACA | |
| AGAGACGTGGCCGGGACCCTGAGATGG | |
| GGGGAAAGCCGCAGAGAAGGAAGAACC | |
| CTCAGGAAGGCCTGTACAATGAACTGCA | |
| GAAAGATAAGATGGCGGAGGCCTACAG | |
| TGAGATTGGGATGAAAGGCGAGCGCCG | |
| GAGGGGCAAGGGGCACGATGGCCTTTAC | |
| CAGGGTCTCAGTACAGCCACCAAGGACA | |
| CCTACGACGCCCTTCACATGCAGGCCCT | |
| GCCCCCTCGCTAA (SEQ ID NO: 7) | |

Cell Lines and Cell Culture

The human T cell Jurkat cell lines, Endogenous TCR and coreceptor deficient Jurkat 76 was a kind gift from Dr. Heemskerk M H and Wild-type Jurkat E6-1 (TIB-152), LCK-deficient Jurkat cam1.6 (CRL-2063), Daudi cell (CCL-213) were from the American Type Culture Collection. The cells were maintained in RPMI-1640 media (Hyclone) supplemented with 10% fetal bovine serum (Hyclone), 2 mM L-glutamine (Gibco) and MEM non-essential amino acid (Gibco) in humidified 5% CO₂ incubator at 37° C. Human embryonic kidney epithelial cells (HEK293) were cultured in DMEM (Hyclone) supplemented with 10% fetal bovine serum (Hyclone), 2 mM L-glutamine (Gibco) and MEM non-essential amino acid (Gibco). Tetracycline-Regulated Expression (T-REx) CHO cell line was purchased from Invitrogen and used for generation of artificial antigen presenting cell line. The CHO cells were cultured in Ham's F-12 (Gibco) medium with 10% fetal bovine serum (Hyclone), 2 mM L-glutamine (Gibco) and MEM non-essential amino acid (Gibco). Transfections of Single-chain trimer MHC constructs into CHO were performed using the polyethylenimine (PEI) method. After transfection, the cells were selected by the addition of drugs (1.0 mg/ml G418 sulfate from Hyclone). Single cell sorting was then applied to select a proper HLA expression clone. The pMHC complex expression was checked regularly by flow cytometry.

Antibodies and Chemicals

The following antibodies were used in this study: Myc-Tag mouse mAb Alexa Fluor 647 (9B11), anti-pSrc family (pY416), anti-pPLCγ1, anti-p44/42 Erk1/2, anti-Erk1/2 (All from Cell signaling Technology); rabbit anti-human FYN (FYN-59), anti-Human CD28 Alexa 488 (CD28.2), anti-human CD80 PE (2D10), anti-human CD19 FITC, anti-human CD86 Brilliant Violet 421 (BU63), anti-human CD3 APC, anti-human CD279 (PD-1) PE (All from Biolegend); anti-p CD3ζ (pY142), mouse anti-human c-CBL, anti-PLCγ1 (All from Becton Dickinson); anti-human HLA-A2 APC (BB7.2), anti-human CD8A APC, anti-human CD8B PE-Cy7 (All from eBioscience); mouse anti-human LCK (3A5, Santa Cruz Biotechnology); goat anti-mouse IgG (H+L) secondary antibody Alexa Fluor 647 (Thermo Fisher Scientific); specific HLA/A2-L2, HLA/A2-L1. HLA/A2-E1 TCR-like antibodies were produced as described (Sim et al., 2013). For chemicals used in this study: SRC family kinase (SFK) inhibitor PP2 was purchased from Sigma-Aldrich; specific LCK or FYN inhibitor (A770041 or SU6656, respectively) was from MedChemExpress or SelleckChem, respectively; the calcium dye Indo-1, AM was from Thermo Fisher Scientific.

Lentivirus Production and Transduction

A total of 6.5×10⁵ HEK293 cells per well were seeded onto 6 well plates one day before transfection and incubated at 37° C. with 5% CO₂. The cells were then transfected with packaging plasmid and lentiviral vector using polyethylenimine (PEI), and medium was replaced after 12 hr. The viral supernatant was harvested twice on the following two days. The collected viral supernatant was titred, filtered by 0.45 um membrane filter (Millipore) and concentrated 100× by ultracentrifuge tube (Millipore). For lentivirus transductions, polybrene and HEPES were added at 8-10 ug/ml and 10 mM respectively with Jurkat cells at 1×10⁶ per well in 1 ml, followed by spinoculation at 2,500 rpm for 2 hrs. For CHO cell transduction, viral solution was directly added with the cell without spinoculation. After 24 hrs, cells and viral solution were separated, and cells were cultured in the maintenance medium. After an additional 48 hrs culture period, flow cytometry analysis was performed to check expression of the constructs.

Electroporation

The electroporation was performed based on instruction of Amaxa cell line nucleofector kit (VCA-1003). In brief, 10⁶ cells for each sample were prepared, and spun down at 90×g for 10 mins. The cells were resuspended the cell with 100 ul nucleofector solution and combined with 2 g DNA. The electroporation program X-05 (high efficiency) was applied in Nucleofector® 2b Device. The expression of the construct was detected by flow cytometer after 18 hrs.

Imaging

For total internal reflection fluorescence microscopy (TIRFM), lipid bilayers containing specific pMHC and other anchoring proteins were prepared as previously described. In brief, 0.2 mol % liposomes were prepared, evaporated under N2 at 37° C., and sonicated for yielding 4 mM lipid stock. Glass 8-well chamber LabTekII chamber slides (Fisher Scientific) were cleaned in 6 M NaOH for 2 h and rinsed with ddH₂O before adding the lipid. The lipids were diluted 10-folds in PBS, added to clean chamber slides and incubated for 30 mins. Excess liposomes were washed away with 12 ml PBS. The bilayers were blocked with 2 mg/ml BSA for 30 min. 5 μg/ml streptavidin was added and incubated for 30 mins. After washing the excess streptavidin, biotinylated HLA/A2-L2 monomer, recombinant human ICAM1 Protein, hIgG1-Fc.His Tag (Thermo Fisher Scientific) were added to the bilayers for 30 min. After washing, the bilayers were ready to use. Per 10⁵ cells/100 μl of CAR-Jurkat or TCR-Jurkat with CD8α-mCherry were added onto one well of the chamber at 37° C. for 10 mins. 100 μl of 8% paraformaldehyde was then added to fix the cell and stop the stimulation. TIRF microscopy was later performed on an Olympus IX83 inverted microscope fitted with a four-laser TIRF module. Images were acquired using a 40×/1.49 NA oil-immersion lens. Fluorescence excited within the 100-nm evanescent field was recorded with a Hamamatsu ORCA Flash 4.0 camera. For regular fluorescent imaging, 10⁵ cells of CAR-Jurkat or TCR-Jurkat with CD8α-mCherry were mixed with 10⁵ cells specific CHO-APC in 100 μl RPMI medium for 10 mins on one well of the chamber. This was followed by adding 100 μl of 8% paraformaldehyde. CD8 recruitment was later detected on an Olympus IX83 inverted microscope in normal module. Images were acquired using a 20×-40×/1.49 NA oil-immersion lens.

Calcium Flux Assay

Cell samples were suspended at a density of 10×10⁶ cells per ml in phosphate-buffered saline (PBS) and loaded with 2 uM Indo-1 AM for 30 min at 37° C. incubator, followed by washing twice with culture RPMI. Cells were pre-warmed to 37° C. for 10 min before analysis and were kept at 37° C. in culture RPMI during the event collection. For cell stimulation, an HLA/A2-L2 monomer was pre-refolded, biotinylated and crosslinked with streptavidin Alexa 647 (Thermo Fisher) to form antigen tetramer. Cells were then stimulated with tetramers. Mean fluorescence ratio of Indo-1 high (BUV395)/Indo-1 low (DAPI) was calculated with FlowJo by kinetics program.

T Cell Stimulation Assay

Artificial antigen presenting CHO cells (APC-CHO) were seeded onto a 96 well flat plate at 2-3×10⁴ per well one day before. For APC-CHO requiring peptide pulsing, peptide was added at 4 uM for 3 hrs, and then washed away before CAR-T or TCR-T cells were added. Each CAR-T cell or TCR T cell sample was counted and suspended at a concentration of 10⁶ per ml in culturing RPMI medium. 200 ul per well of suspended cell solution was then added into APC-CHO pre-seeded plate. For inhibitor experiments, inhibitor was added accordingly into the cell mixture. Technical triplicates were performed for all experiments. The cells were incubated at 37° C., 5% CO₂ for 18 hrs. After incubation, supernatant was collected for human IL-2 ELISA assay, which was performed according to the manufacturer's protocol (Invitrogen). The T cell pellets were resuspended for surface staining or used for another round of stimulation by repeating the method described above.

Western Blotting

A total of 10⁶ cell samples were lysed by NP-40 lysis buffer. Cell debris was pelleted, and supernatant was collected and heated with reduced protein loading buffer (Thermo Fisher Scientific). To detect phosphorylation after stimulation, 10⁵ APC-CHO cells were seeded per well in a 24-well plate one day before stimulation. 10⁶ CAR-T or TCR-T cells were added into each well and incubated at 37° C., 5% CO₂ for designated duration. The stimulated CAR-T or TCR-T cells were collected and prepared as above. The samples were loaded in a 4-12% Bis-Tris gradient gel (NuPAGE, Invitrogen) and transferred to a PVDF membrane (Immobilon-FL Transfer Membrane, Millipore). The membrane was then blocked using blocking buffer (Odyssey, LI-COR) for 1 hr at room temperature. Subsequently, the membrane was probed with different primary antibodies. The secondary antibodies used were IRDye 800CW Goat anti-Mouse IgG2b (Cat #926-32352, LI-COR) and IRDye 680LT Goat anti-Rabbit (Cat #926-68021). The blotting was quantified by the LI-COR Odyssey infrared imaging system.

CRISPR-Cas9 Genetic Editing

The Cas9 plasmid was obtained from Addgene (#52961). The gRNA sequences for FYN and LCK were retrieved from http://chopchop.cbu.uib.no/and are shown in Table 2. Cas9 sequence was linked with mTagBFP by P2A cleavable linker and then cloned together with gRNA sequence into lentiviral vector. After transduction into Jurkat cells, single cell sorting was followed by mTagBFP fluorescent marker. Intracellular staining and western blotting were done for clone screening.

TABLE 2
gRNA pool for LCK and FYN knock out. Each of gRNA
was selected from http://chopchop.cbu.uib.no/.
After screening, LCK gRNA2 and FYN gRNA3 were
chosen for LCK and FYN knock out respectively.

| gRNA | Sequence | |
|---|---|---|
| LCK gRNA1 | GACCCACTGGTTACCTACGA | (SEQ ID NO: 9) |
| LCK gRNA2 | GCCGGGAAAAGTGATTCGAG | (SEQ ID NO: 10) |
| LCK gRNA3 | CGGGACTTCGACCAGAACCA | (SEQ ID NO: 11) |
| LCK gRNA4 | TGGAGCCCGAACCGTAAGTG | (SEQ ID NO: 12) |
| LCK gRNA5 | CGGCGGATTGGAGCCTTCGT | (SEQ ID NO: 13) |
| LCK gRNA6 | GCGGACCAGTTCATGCAGGC | (SEQ ID NO: 14) |
| LCK gRNA7 | CGTTGTAGTACCCTGGGTCG | (SEQ ID NO: 15) |
| LCK gRNA8 | GGGTGACAATTTCCGTCAGC | (SEQ ID NO: 16) |
| LCK gRNA9 | CGTAAGTGGGGACCCGTCG | (SEQ ID NO: 17) |
| LCK gRNA10 | CTGGAGCCCGAACCGTAAGT | (SEQ ID NO: 18) |
| LCK gRNA11 | GGTGAATGTCCCGTAGTTAA | (SEQ ID NO: 19) |
| LCK gRNA12 | GATCGTTTTCACTGTCGGTC | (SEQ ID NO: 20) |
| LCK gRNA13 | CCGTAAGTGGGGACCCGTCG | (SEQ ID NO: 21) |
| LCK gRNA14 | CCCTCTCACGACGGAGATCT | (SEQ ID NO: 22) |
| LCK gRNA15 | CTGGCGCCCGGGAACACTCA | (SEQ ID NO: 23) |
| LCK gRNA16 | GTAAGTGGGGACCCGTCGTG | (SEQ ID NO: 24) |
| LCK gRNA17 | TGTGCCCGTTGTAGTACCCT | (SEQ ID NO: 25) |
| LCK gRNA18 | ATTACACCAGTGAGCCCGAC | (SEQ ID NO: 26) |
| LCK gRNA19 | TGGCCTAGCACGCCTCATTG | (SEQ ID NO: 27) |
| LCK gRNA20 | CGTAGAGCCGAACCAGCCGC | (SEQ ID NO: 28) |
| LCK gRNA21 | ATCCGTAATCTGGACAACGG | (SEQ ID NO: 29) |
| LCK gRNA22 | GCCCTCTCACGACGGAGATC | (SEQ ID NO: 30) |
| LCK gRNA23 | ATCGTTTTCACTGTCGGTCC | (SEQ ID NO: 31) |
| LCK gRNA24 | GAACCTGAGCCGCAAGGACG | (SEQ ID NO: 32) |
| LCK gRNA25 | CCATTATCCCATAGTCCCAC | (SEQ ID NO: 33) |
| LCK gRNA26 | GCTCACACCCGGAAGATGAC | (SEQ ID NO: 34) |
| LCK gRNA27 | CTCACGGCTCCTTCCTCATC | (SEQ ID NO: 35) |
| LCK gRNA28 | AAGGCGCAGTCCCTGACCAC | (SEQ ID NO: 36) |
| LCK gRNA29 | CTACGAAGGCTCCAATCCGC | (SEQ ID NO: 37) |
| FYN gRNA1 | AGAGTTCACACCTCCAAAGA | (SEQ ID NO: 38) |
| FYN gRNA2 | ACGGGGACCTTGCGTACGAG | (SEQ ID NO: 39) |
| FYN gRNA3 | TTGTCCTTTGGAAACCCAAG | (SEQ ID NO: 40) |
| FYN gRNA4 | GTCCCCCGAATCATTCCTTG | (SEQ ID NO: 41) |
| FYN gRNA5 | TGGATACTACATTACCACCC | (SEQ ID NO: 42) |

Flow Cytometry and Cell Sorting

Flow cytometry experiments were conducted on BD LSR Fortessa X-20 (Becton Dickinson). Cell sorting was conducted on either Mo-flo XDP (Beckman Coulter, Inc.) or SY3200 (Sony Biotechnology Inc.) by Flow Cytometry Laboratory, Immunology Programme, National University of Singapore. Data analysis was performed on FlowJo.

Statistical Information and Data Analysis

Two-tailed Student t test or two-way ANOVA analysis was performed for column data or curve data respectively by using GraphPad Prism 7. The data meet the assumptions of the tests. Variance is similar between the compared groups.

### Example 1

Artificial Antigen Presenting CHO Cell Provides Antigens for CAR and TCR T Cell Stimulation

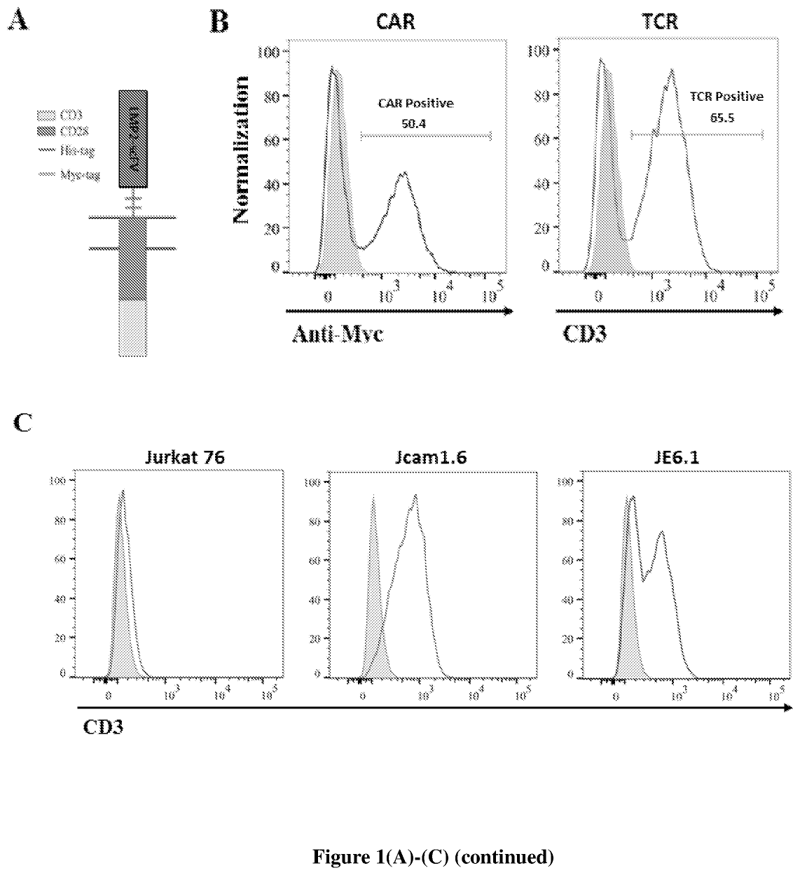

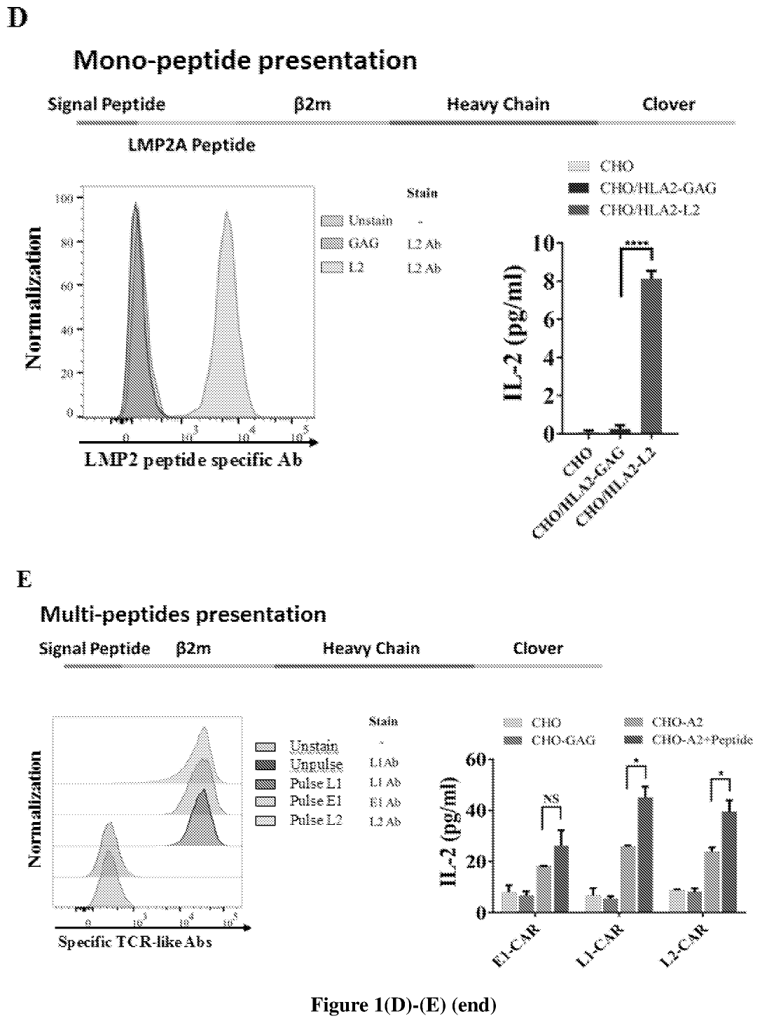

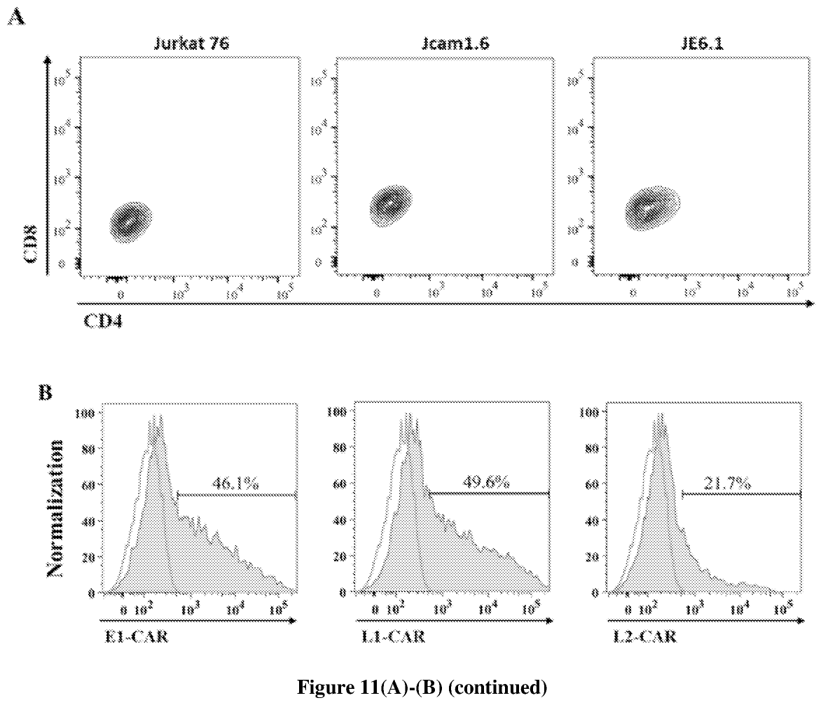

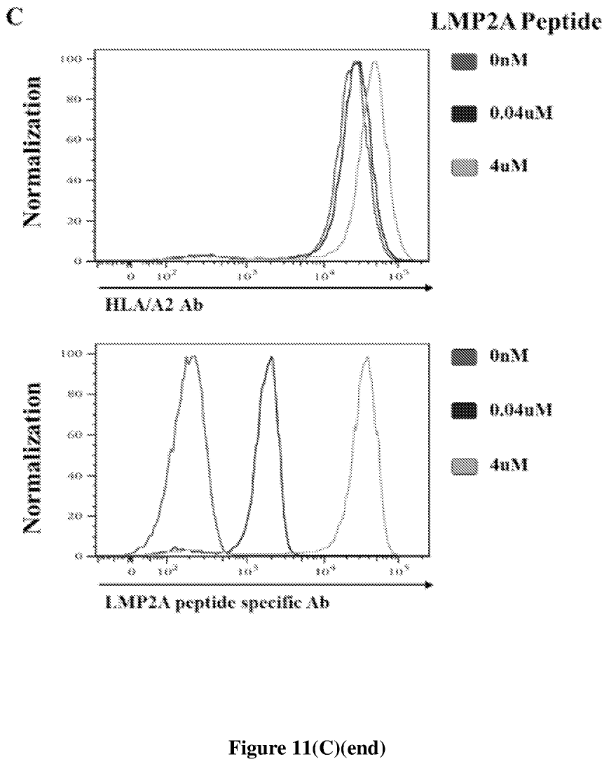

To compare molecular recognition between CAR and TCR in detail, Jurkat T cells expressing CAR and TCR with the same peptide-MHC specificity were generated. The CAR construct was generated based on a previous report where CD28 costimulatory domain was described (Figure. 1A). The scFv on CAR was constructed from a TCR-like antibody, recognizing a peptide epitope of Latent membrane protein 2A (LMP2A) protein from Epstein-Barr virus (EBV) presented by HLA-A2. The term CAR may refer to this second generation CAR using the CD28 transmembrane and cytoplasmic domains. Lentivirus was then applied to deliver CAR into Jurkat 76 T cells, which lack endogenous TCRα and β chains, but contain the full set of CD3 subunits. The lentivirus transduction was efficient both for CAR or for the TCR specific for the same peptide-MHC complex as CAR (FIG. 1B). CAR or TCR-expressing Jurkat cells were sorted to generate stable CAR or TCR-Jurkat cells. There are various human Jurkat T cell lines available for T cell signaling studies. Here, three Jurkat-derived cell lines, Jurkat 76, JE6.1, and Jcam1.6 were used. Jurkat 76 were used for the majority of experiments, thus they are referred to as Jurkat unless otherwise stated. Only Jurkat 76 did not express CD3 on the surface, but JE6.1 and Jcam1.6 expressed different amounts of CD3 (FIG. 1C). None of these three cell lines express either CD8 or CD4 (FIG. 11A). In addition, an artificial antigen presenting system based on expression of human MHC class I in single chain format in xenogeneic CHO cells was also engineered. This antigen presenting cell (APC) system was built into two versions, a mono-peptide system where a peptide of choice is covalently fused to β2-microglobulin and heavy chain (FIG. 1D), and a multi-peptide system where the peptide groove of HLA-A2 is open and multiple peptides can be pulsed onto the cells to bind to the MHC-I molecule (FIG. 1E). Both system presented peptides efficiently, as observed that specific peptide presentations were significantly detected by specific TCR-like antibodies respectively contrasting with the irrelevant peptide presentations (FIG. 1D,E). Nonetheless, the CAR-T cells reacted differently upon stimulation by these systems as seen in FIGS. 1D and E. LMP2A (L2) peptide-specific CAR-T would specifically respond to CHO-L2, but not irrelevant CHO-GAG. However, the TCR-like CAR-T cell showed some non-specific response against unpulsed CHO cells expressing HLA-A2 (FIG. 1E). This non-specific activation was also found in TCR-like CAR with other specificities, like EBNA1 peptide-targeted E1-CAR or LMP1 peptide-targeted L1-CAR. As detected by anti-HLA-A2 (BB7.2) at different peptide concentrations, the HLA-A2 construct is expressed without deliberate pulsing of peptide onto the CHO-APC (FIG. 11C). Therefore, various unknown peptides from CHO cell are presented in the multi-peptide system. Some of the peptide sequences might resemble the specific peptides, causing non-specific triggering.

Activation of TCR-Like CAR is not Enhanced by CD8 and can Transduce T Cell Signaling without LCK

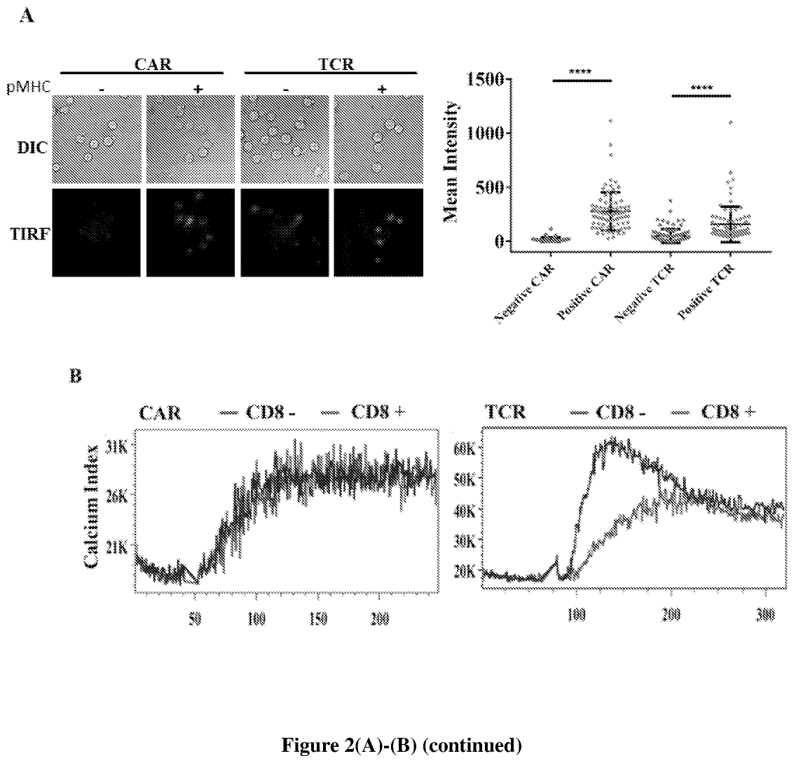

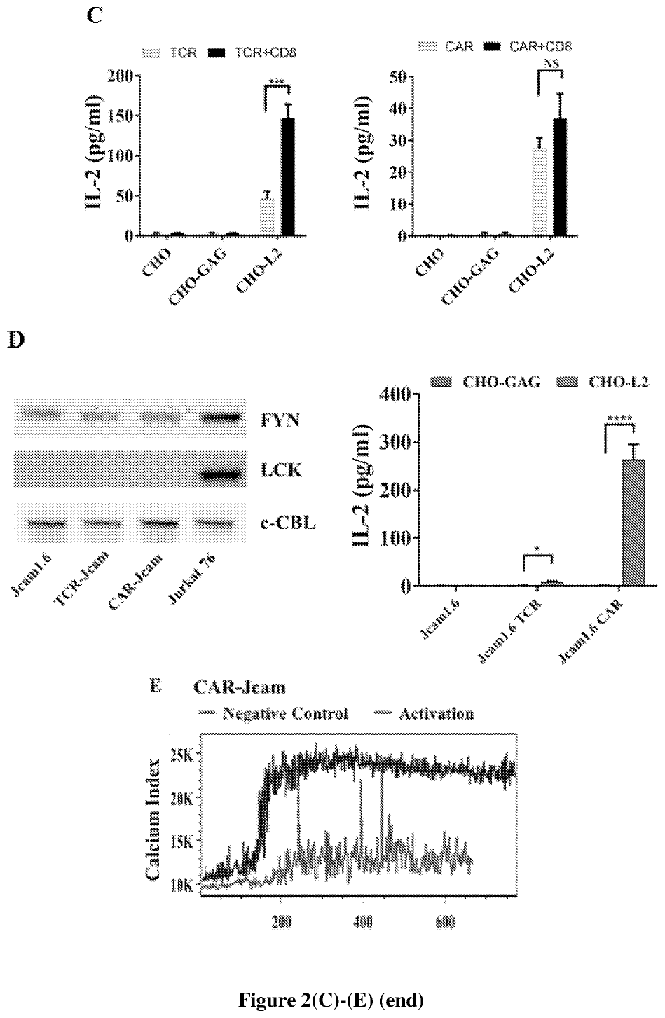

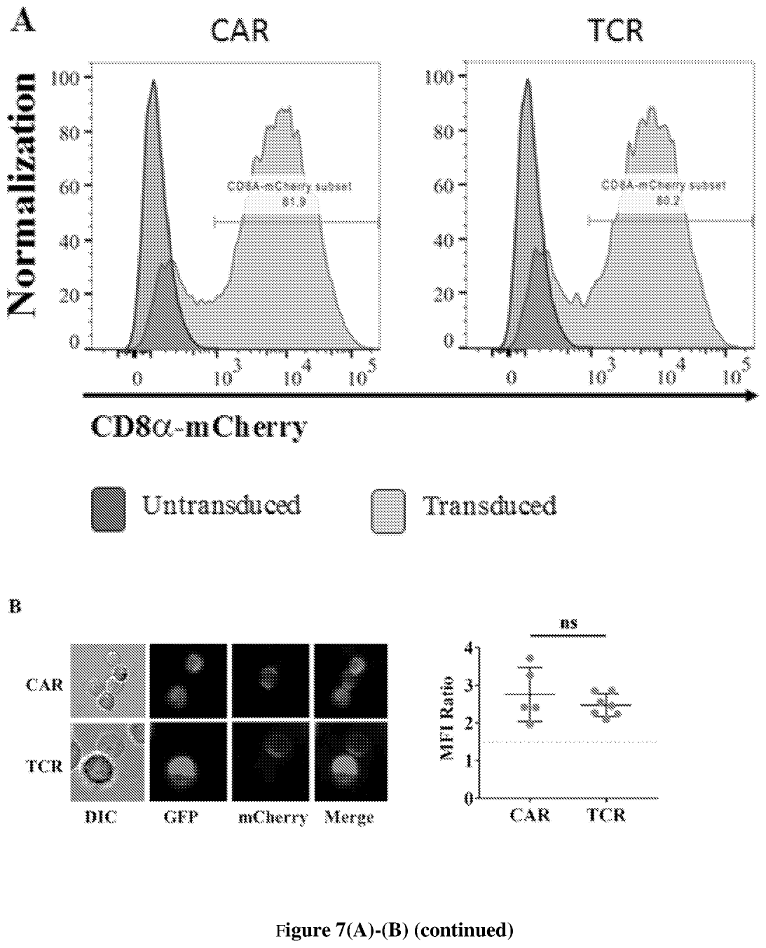

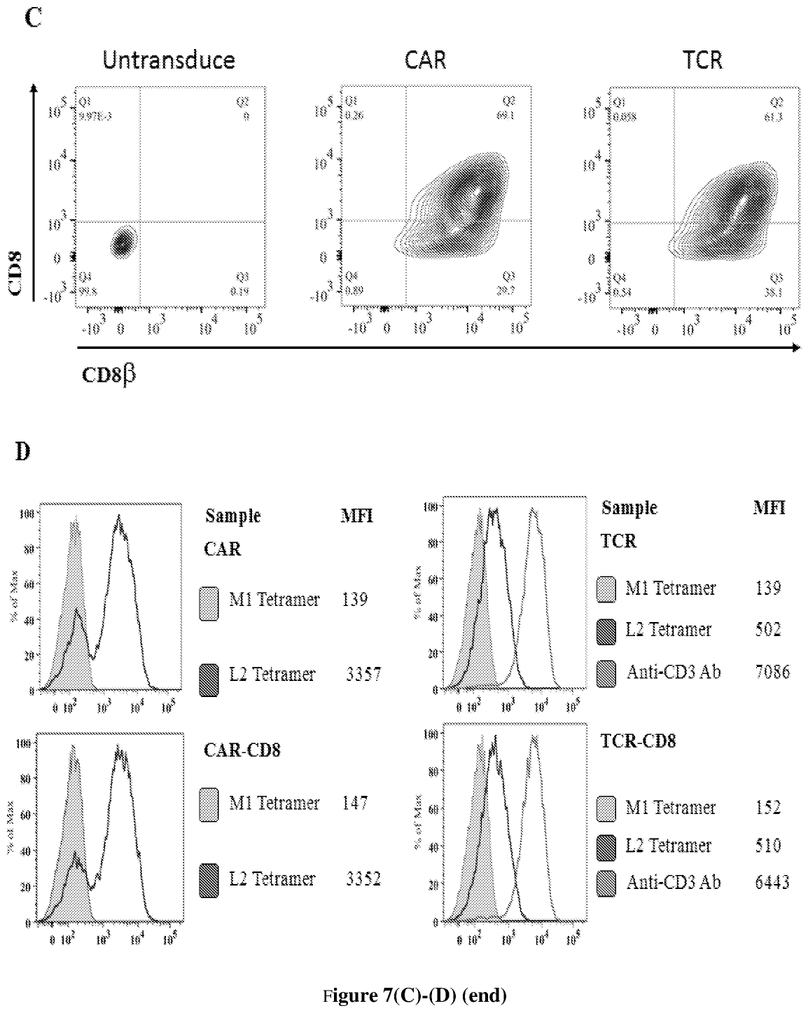

Since this TCR-like CAR has the same specificity for peptide-MHC as the TCR, it is of great of interest to identify the contribution of CD8 coreceptor to T cell activation. It was firstly showed that CD8 coreceptor could be recruited by CAR-T cells as it is in TCR-T cells, using total internal reflection fluorescence microscopy (TIRFM), which was used to detect events at the contact surface (FIG. 2A). CD8α was labelled as a chimera with the fluorescent protein mCherry. Both CAR-T and TCR-T expressed CD8α-mCherry on the surface (FIG. 7A). For TIRFM, a supported lipid bilayer was prepared on a glass plate, and the integrin ligand ICAM-1 was added to anchor CAR-T or TCR-T to the detecting surface. After CAR-T or TCR-T cells expressing CD8α-mCherry were added onto the bilayer with or without specific pMHC for 10 mins, the mean fluorescence intensity (MFI) significantly increased in both groups where pMHC had been added. Clustering of CD8α-mCherry within the contact surface of CAR-T and TCR-T with the bilayer was clear. The MFI ratio between inside and outside the immune synapse was calculated using regular widefield fluorescent microscopy (FIG. 7B). No significant difference was detected between CAR-T and TCR-T with CD8α-mCherry, and both MFI ratios exceeded our cut-off ratio definition for immune synapse formation, 1.5, demonstrating that CD8α-mCherry was recruited in both CAR-T and TCR-T. It was next sought to identify whether CD8 would contribute to CAR-T functionally, given that CD8 could be recruited. After CD8α and CD80 were co-transduced into both CAR- and TCR-Jurkat cells, and CAR or TCR expression amount was as shown to be similar (FIG. 7C,D), it was found that, as expected, the reactivity of TCR-Jurkat bearing the CD8αβ coreceptor was significantly increased. The TCR-T with CD8 showed faster and stronger calcium flux than that of TCR-T without CD8 (FIG. 2B). IL-2 produced by TCR-T with CD8 coreceptor was nearly 3-fold higher than that without CD8 coreceptor (FIG. 2C). However, the reactivity of CAR-Jurkat bearing CD8 was not enhanced over cells lacking CD8 (FIG. 2B,C). The calcium flux of CAR-T with and without CD8 were equivalent, and no significant increase of IL-2 production was detected in CAR-T with CD8. CD8 is considered to be important in enhancing TCR signaling by bringing LCK into the immune synapse. It was then hypothesized that LCK might not be so crucial for CAR signaling, at least as regards CD8-bound LCK. Surprisingly, CAR-T was even able to activate TCR signaling without LCK present, as observed when LCK deficient Jurkat cell line Jcam1.6 was used (FIG. 2D,E). The CAR-Jcam cells could produce IL-2 and fluxed calcium normally, whereas the TCR-Jcam cells were unable to produce IL-2 upon stimulation by antigen.

### Example 2

LCK-Independent CAR Signaling Requires CD28 Costimulatory Domain

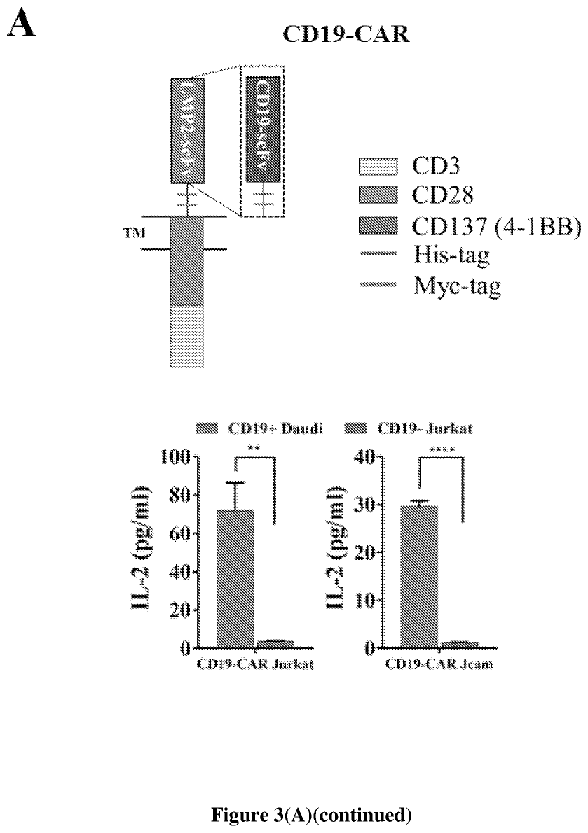

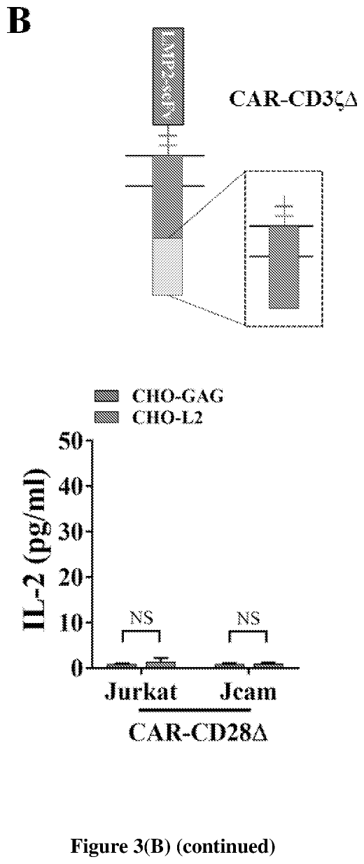

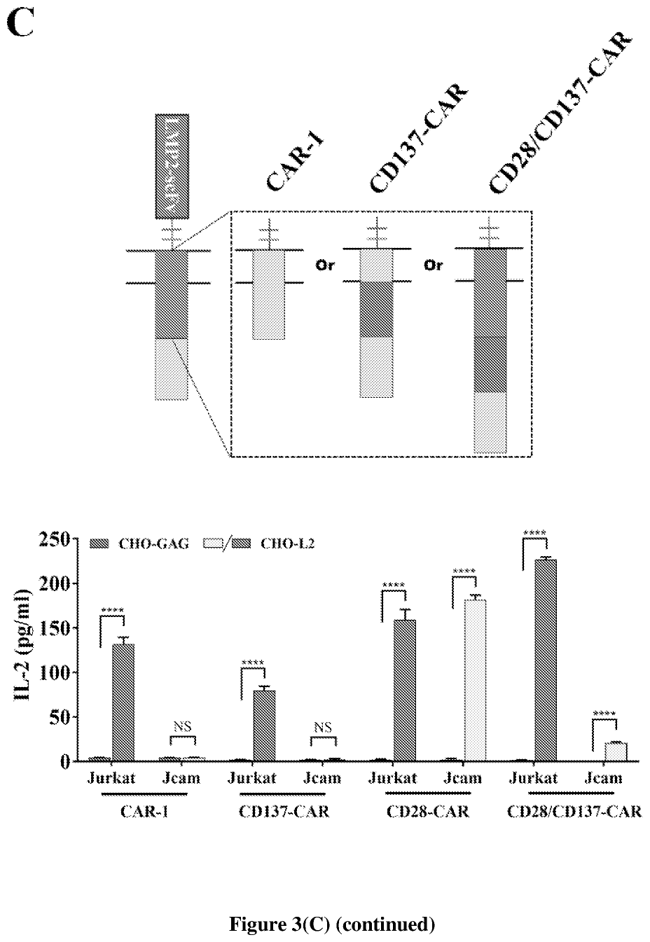

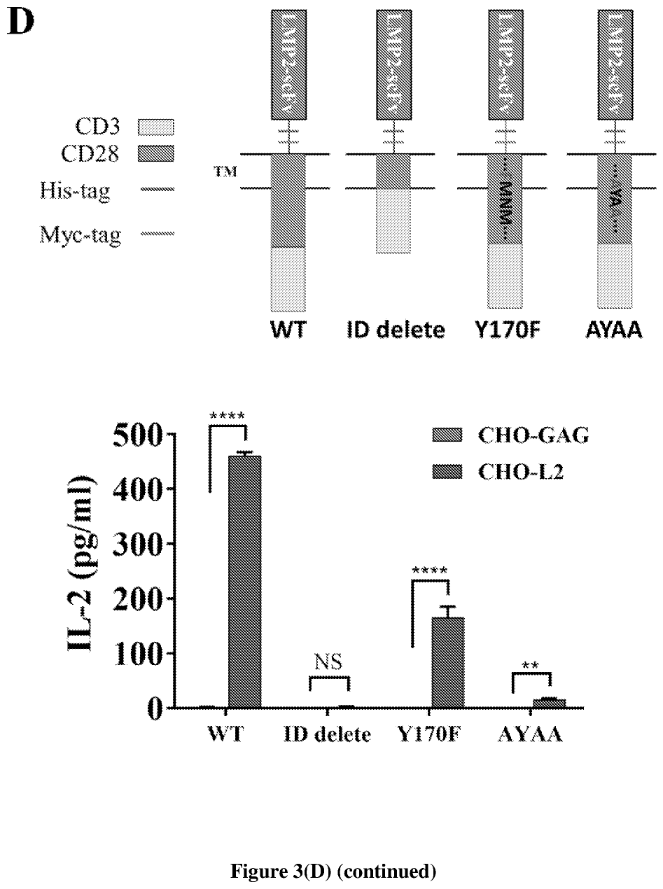

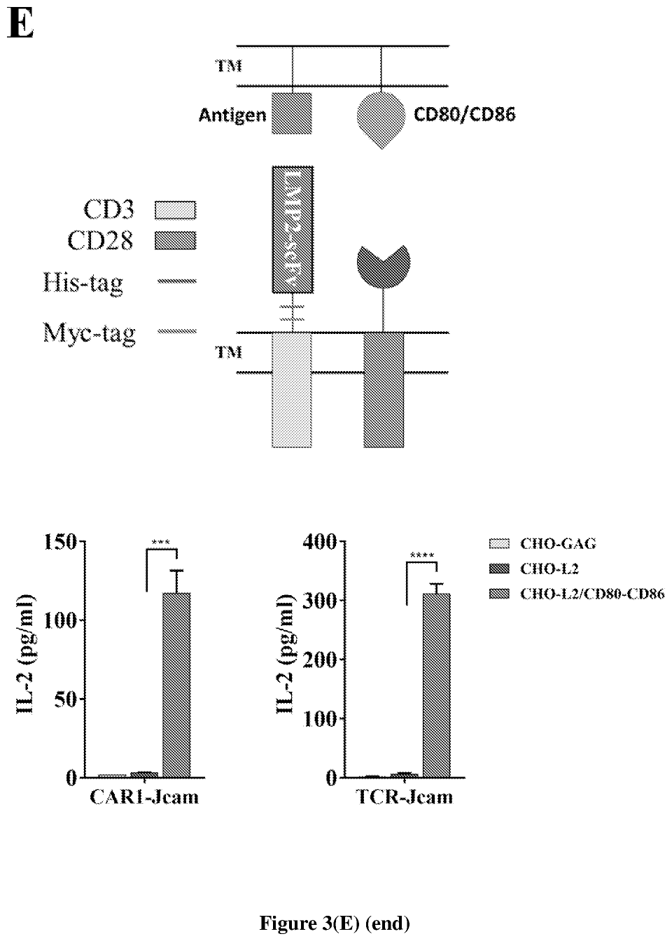

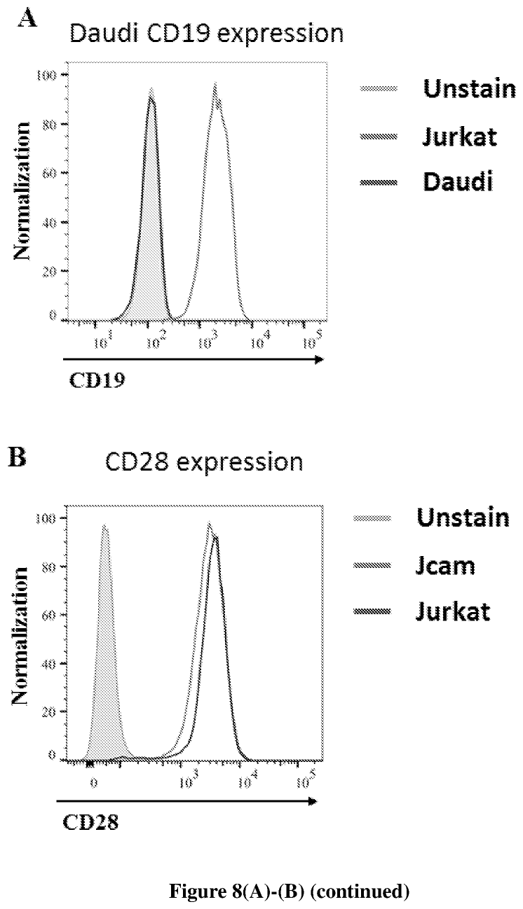

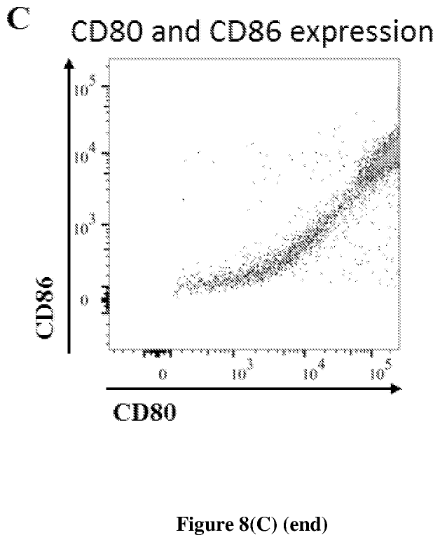

Systematic domain replacements were performed on the CD28-bearing second generation CAR in order to locate which domains trigger the non-canonical T cell signaling of CAR in the absence of LCK. Firstly, it was tested if LCK-independent signaling results from the antigen specificity of the extracellular domain. The TCR-like scFv was swapped to a CD19-scFv, and a CD19-expressing Daudi cell line was used as a target cell (FIG. 8A). The CD19-specific CAR-T cells were activated by the CD19-expressing Daudi cells in the presence or absence of LCK, suggesting that LCK-independent CAR signaling is independent of the antigen specificity of the extracellular CAR domain (FIG. 3A). To determine the importance of the CD3ζ domain, the CD3ζ intracellular domain was deleted, and no IL-2 production was found, demonstrating the indispensability of the CD3ζ ITAMs in CAR-T signaling (FIG. 3B). It was then sought to determine the role of co-stimulatory signaling domains in mediating LCK-independent CAR triggering by: 1) deleting the costimulatory CD28 domain to create a first generation CAR, 2) replacing CD28 with a CD137 (4-1BB) intracellular domain, or adding the CD137 domain to make a third generation CAR (FIG. 3C). The first-generation CAR-1 without a costimulatory domain behaved like TCR, and the signaling was abolished when CAR-1 was introduced into LCK-deficient Jcam1.6 cells. CD137-CAR was constructed. The results show that, for the second generation CARs, only in the design containing the CD28 costimulatory domain was signaling LCK-independent. Nonetheless, in a third generation CAR containing both CD28 and CD137 domains, CAR-T activation in response to antigen was observed under LCK-deficient conditions, but was not as strong in the absence of LCK as that of the CAR containing only the CD28 intracellular domain (FIG. 3C). The involvement of the CD28 signaling pathway was substantiated by mutations of functional binding motifs in the CD28 intracellular domain. The PI3K binding motif and proline rich region were tested as they were reported to be critical in CD28 signal transduction (FIG. 3D). Three mutations were made in the CAR construct: intracellular domain deletion; the PI3K binding motif YMNM was mutated to FMNM; the prolines in the proline rich region PYAP were replaced by alanines. All three mutants were then transduced into the Jcam1.6 cell line. IL-2 production was totally abrogated without the CD28 intracellular domain. Reduced IL-2 production was observed in both FMNM and AYAA mutants, where the AYAA mutant decreased the activation more than FMNM mutant. To further test the influence of CD28 signaling in LCK-independent signaling, CD80 and CD86 were co-transduced into CHO-L2 to activate CD28 signaling. Expression of endogeneous CD28 was identified both in Jurkat and Jcam1.6 (FIGS. 8B,C). Surprisingly, CAR-1-Jcam and TCR-Jcam cell showed restored IL-2 production when CD80 and CD86-expressing CHO-L2 cells were used to stimulate CAR-1-Jcam or TCR-Jcam cell. These results indicate that LCK-independent CAR signaling requires CD28 costimulatory signal, mediated at least partly though YMNM and PYAP motifs, either from CD28 intracellular domain present on second generation CAR or from endogenous CD28 molecule when first generation CAR or TCR was used.

CD28-CAR Relies on FYN to Transduce Downstream Signaling

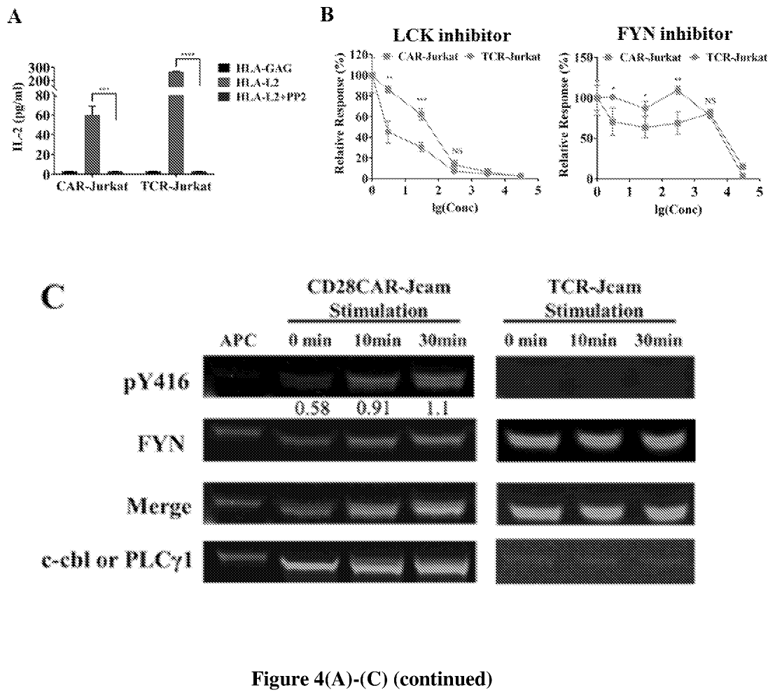

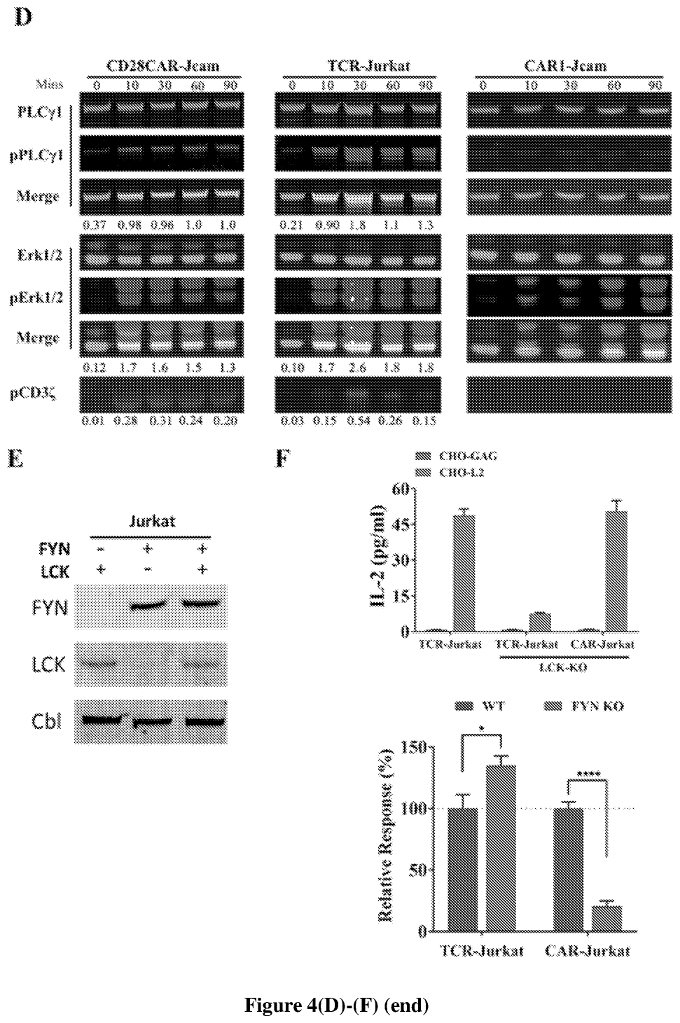

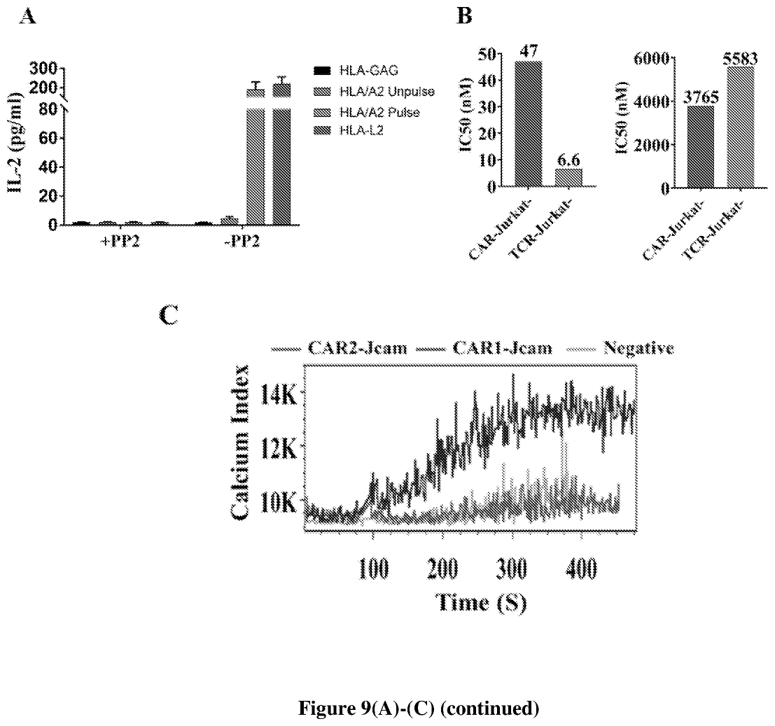

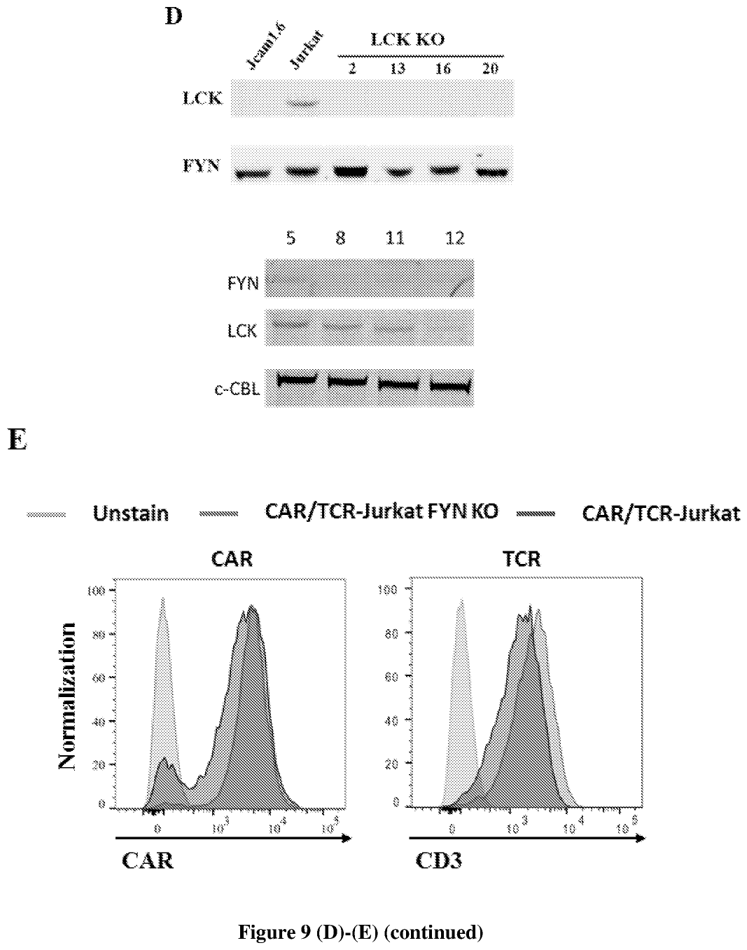

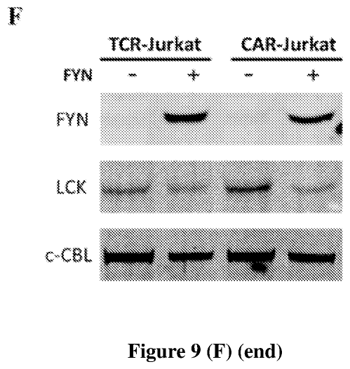

The inventors next tried to identify what kinase was responsible for phosphorylation of CAR during signaling in the absence of LCK. The IL-2 production of CAR-Jurkat or CAR-Jcam was entirely quenched after SRC family kinase (SFK) inhibitor PP2 was added (FIG. 4A, FIG. 9A) showing that CAR signaling is critically dependent on SFKs. Given that LCK and FYN are the most prominent and relevant SFKs in T cells, specific inhibitors A770041 and SU6656 targeting LCK and FYN, respectively, were used to test the relative role of these two SFKs in CAR and TCR signaling. As shown in FIG. 4B, TCR-Jurkat was more sensitive than CAR-Jurkat to the LCK inhibitor. Conversely, CAR-Jurkat was more sensitive than TCR-Jurkat to the FYN inhibitor (FIG. 4B). The IC50 of the inhibitors towards CAR-Jurkat or TCR-Jurkat further substantiated the disparate sensitivity; the IC50 of the LCK inhibitor was 6.6 nM for TCR-Jurkat, but 47 nM for CAR-Jurkat. For the FYN inhibitor, IC50 on CAR-Jurkat (3765 nM) was lower than that (5583 nM) to TCR-Jurkat (FIG. 9B). This propensity of CAR to preferentially use FYN, unlike TCR that depends on LCK, hinted that FYN could be the kinase activating downstream signaling from the CAR. To further test the involvement of FYN activation in LCK-independent signaling, a western blot was performed. Results of FYN phosphorylation in CAR-Jcam at different timepoints showed that the activation site Y420 of FYN was phosphorylated after CAR-Jcam but not TCR-Jcam engagement with specific APC (FIG. 4C), with nearly two-fold increase after 30 mins in FYN Y420 phosphorylation. The phosphorylation intensity was calculated by the intensity of pY420 to that of total FYN. The activation of downstream key signaling molecules was also explored in LCK-deficient CAR-Jcam (FIG. 4D). Phosphorylation of PLCγ1, Erk, and CD3ζ was detected after activation of CAR-Jcam, whereas only Erk was phosphorylated in CAR1-Jcam. Differences in downstream signal activation between CAR-Jcam and CAR1-Jcam were reflected also in calcium flux experiments (FIG. 9C). CAR-Jcam but not CAR1-Jcam cells showed calcium influx in response to cognate antigen. Different kinetic patterns after activation were also seen between CAR-Jcam and TCR-Jurkat. The phosphorylation of PLCγ1, Erk, and CD3ζ was more stable after CAR signaling than that of TCR. Activation of TCR was a relatively short pulse, as the phosphorylation of each molecule went up at 30 mins and then waned.

To better validate the role of LCK and FYN in CAR and TCR signaling, a CRISPR-Cas9 genetic editing system was introduced to knock-out LCK or FYN. After gRNA screening (Table 2) and single clone sorting (FIG. 9D), LCK knock-out clone 20 and FYN knock-out clone 8 were selected as the LCK or FYN knock-out systems for further experiments (FIG. 4E). Prior to the experiment, the CAR or TCR transduced LCK or FYN knock-out T cells were sorted so that the expression of TCR and CAR was equivalent. In LCK knock-out Jurkat cells, TCR was not able to activate signaling to produce IL-2, but CAR was still functional, consistent with previous results obtained using Lck-deficient Jcam1.6 cell line (FIG. 4F). However, in FYN knock-out Jurkat cells, TCR produced a slightly higher amount of IL-2 than wild-type TCR-Jurkat. However, IL-2 production of CAR-Jurkat FYN KO cell was dramatically reduced compared with the wild-type CAR-Jurkat (FIG. 4F). The expression of CAR or TCR were equivalent in FYN KO Jurkat cells, and lack of FYN was confirmed in CAR or TCR-Jurkat FYN KO by Western blotting (FIG. 9E,F).

### Example 3

LCK-Deficient CAR-T Resets Activation Threshold and Selectively Allows Only CAR Triggering in T Cells Expressing Both CAR and TCR

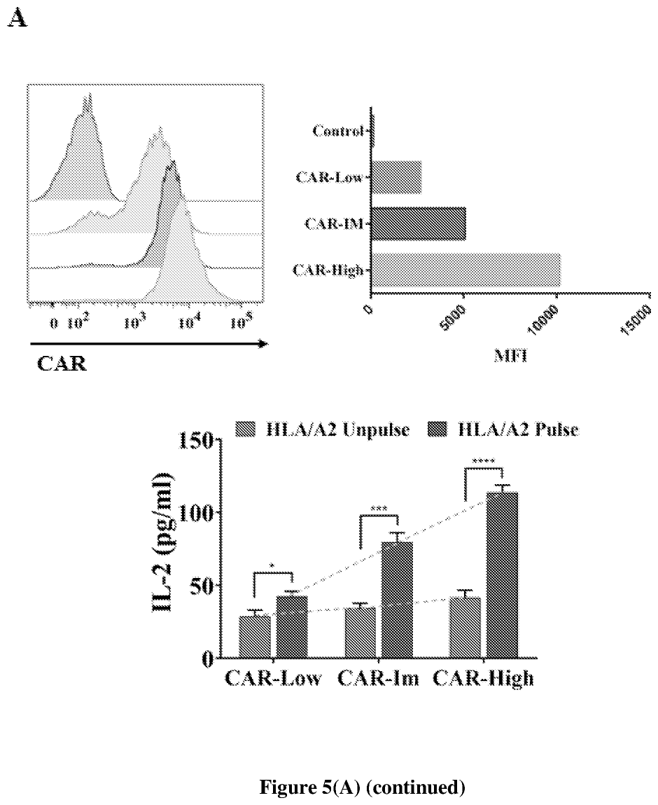

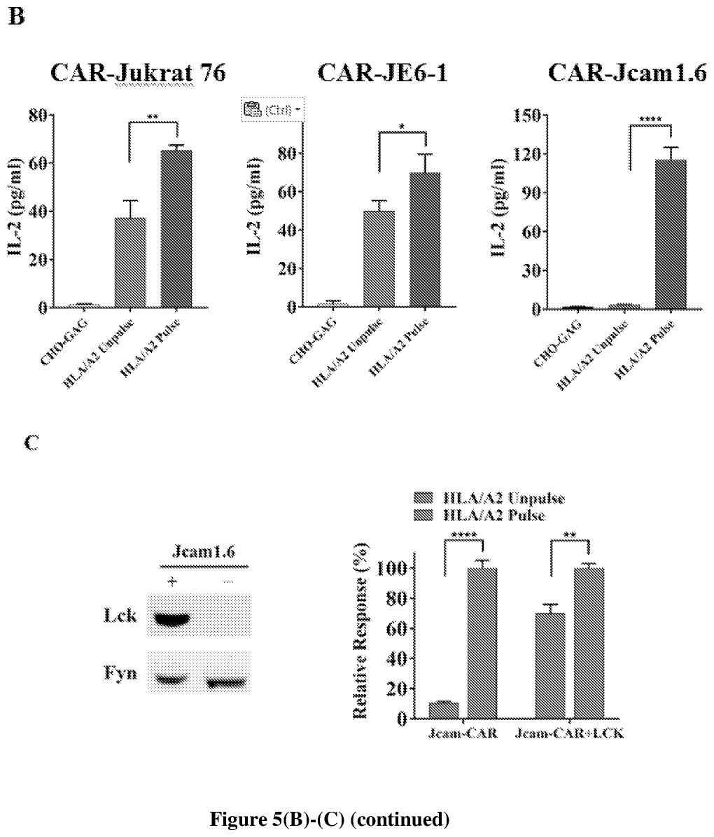

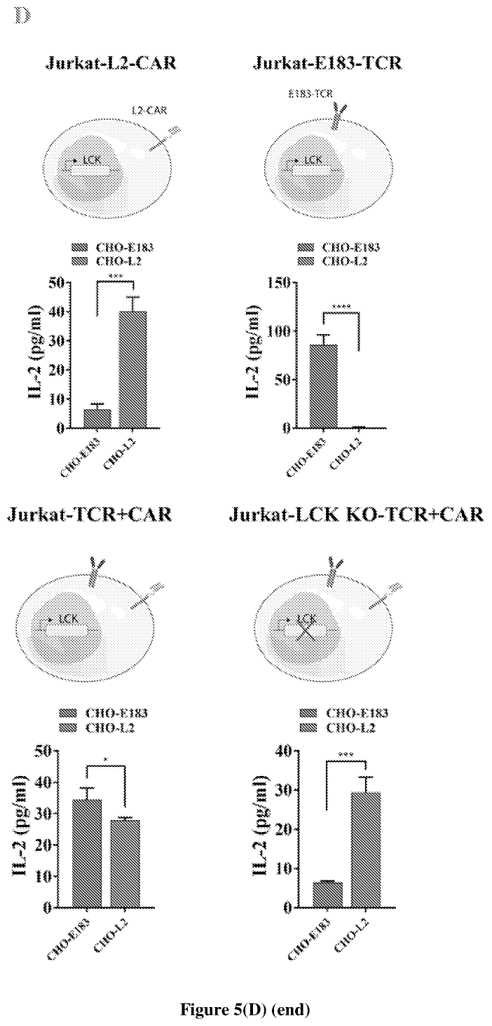

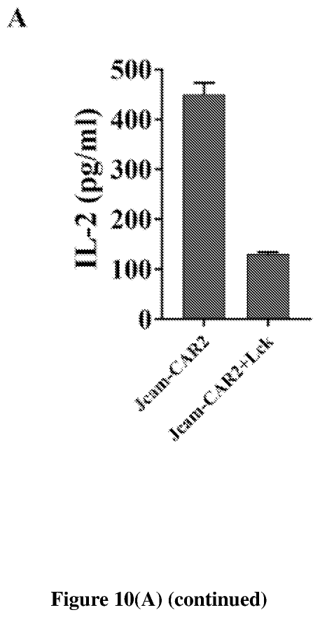

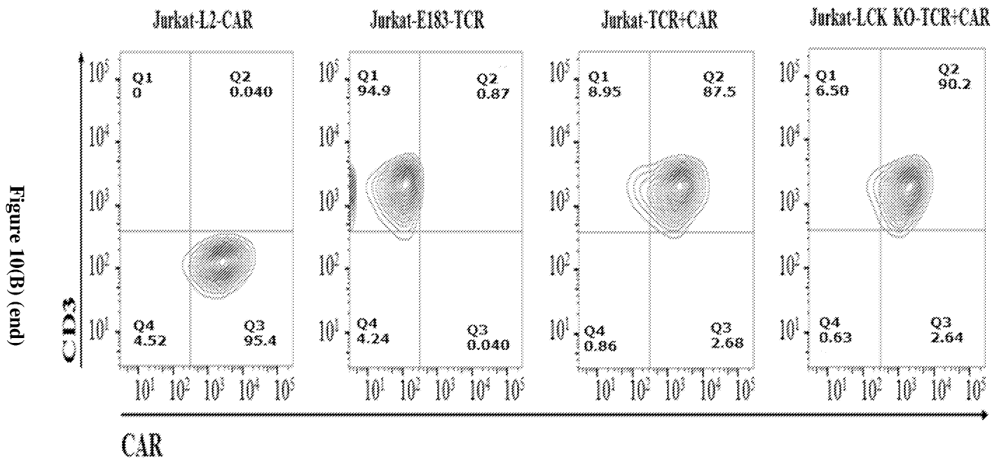

Based upon these findings, it was then sought to investigate potential functional consequences of the LCK-independent triggering mechanisms, and whether LCK-deficient CAR-T cells could have practical applications. The effect of the presence or absence of LCK on CAR activation thresholds was first tested. Some non-specificity in the multi-peptide presenting system (FIG. 1E) was previously noted. This non-specific activation of TCR-like CAR has been reported before, where the specificity could be diminished if the affinity of TCR-like antibody is above a certain threshold. After TCR-like CAR-T cells were sorted for different amounts of CAR expression, it was found that increased amounts of IL-2 secreted from low to high-expressed CAR against unpulsed HLA-A2 or specific peptide-pulsed HLA-A2 was disparate (FIG. 5A). This difference indicated that the TCR-like CAR could have different affinity for non-specific binding and for specific binding, where the affinity of specific binding is higher than that of non-specific binding. It was hypothesized that the activation thresholds would likely be different between LCK-deficient CAR-T and LCK-sufficient CAR-T, given that the activation kinase is distinct in each system. Therefore, responses to the specific and non-specific antigens could be differentiated in LCK-deficient CAR-T. As seen in the comparison among CAR-Jcam, CAR-Jurkat 76 and CAR-JE6-1, IL-2 production by CAR-Jcam was as low as the negative control, which presented irrelevant peptide GAG, whereas the CAR-JE6-1 showed the same non-specific activation as CAR-Jurkat 76 (FIG. 5B). Notably, JE6-1 expresses endogenous TCR like Jcam1.6 (FIG. 1C), thus ruling out a contribution of endogenous TCR to the non-specific activation by CAR. To further validate the role of LCK in mediating TCR-like CAR-T cell activation to the low affinity non-specific antigen, LCK was transduced into Jcam1.6 cells to make a LCK positive CAR-Jcam cell. Again, unpulsed HLA-A2 APC induced strong IL-2 production in LCK positive, but not LCK negative CAR-Jcam cells (FIG. 5C). The IL-2 production against antigenic CHO-L2 was also observed to be lower in LCK-positive CAR-Jcam than in LCK-negative CAR-Jcam cells (FIG. 10A). Moreover, it was predicted that LCK-deficiency would allow selective triggering of CAR, but not TCR, in T cells that express both CAR and TCR. To test this hypothesis, L2 peptide-specific CAR and a TCR with specificity against E183-91 (FLLTRILTI) epitope from Hepatitis B virus (HBV), hence referred as E183-TCR, were co-transduced in Jurkat cells or LCK knock-out Jurkat cells (FIG. 10B). The Jurkat-TCR+ CAR was induced to produce IL-2 by CHO-E183 or CHO-L2, showing that activation of CAR and TCR was unimpaired in dual CAR+TCR system. However, CHO-L2, but not CHO-E183, induced IL-2 production in the LCK-deficient Jurkat T cells co-expressing CAR and TCR, showing that LCK deficiency allows re-wiring of TCR signaling pathways for selective CAR, but not TCR, triggering.

LCK-Deficient CAR-T Cells Express Reduced PD-1 and Show Reduced CAR Downregulation after Stimulation

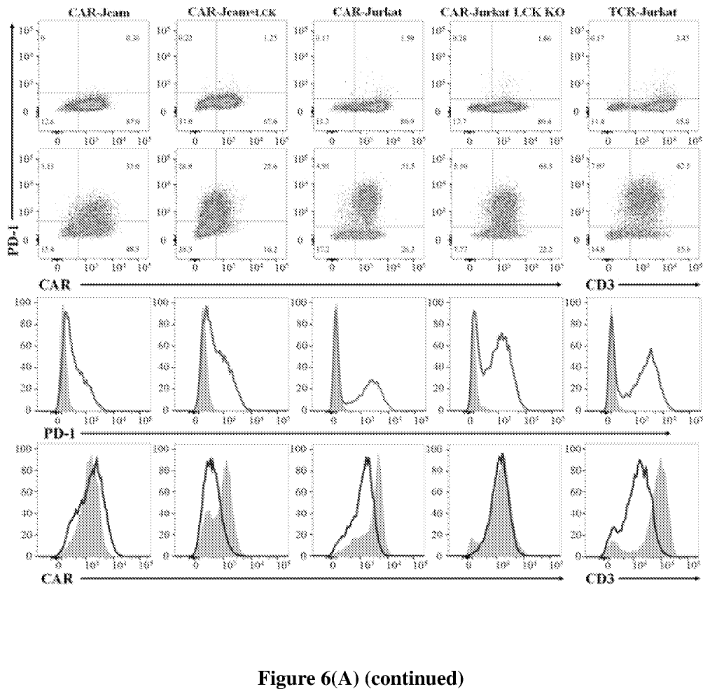

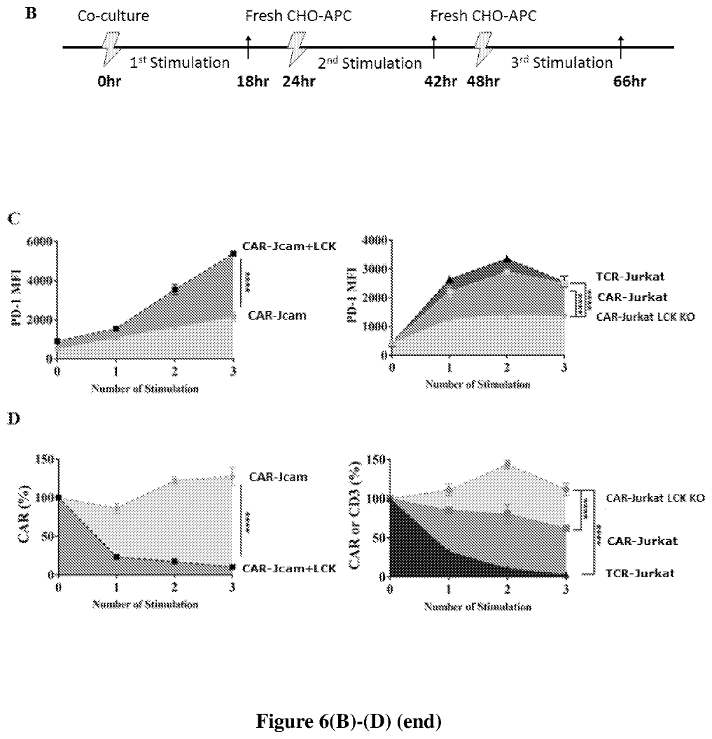

Many changes in gene expression happen after TCR signaling. One of the significant changes is the upregulation of co-inhibitory molecule PD-1, a significant marker of exhaustion in cancer immunotherapy (FIG. 6A). There was no PD-1 expressed on the cell surface before stimulation in either kind of CAR-T cell. The difference was nevertheless obvious after stimulation of the different CAR-T cells. As seen in FIG. 6A, CAR-Jcam transduced with LCK upregulated PD-1 more strongly than LCK_deficient CAR-Jcam. In addition, PD-1 expression was lower in CAR-Jurkat LCK KO than in CAR-Jurkat cells. TCR-Jurkat, however, expressed the highest amount of PD-1 among these groups. These differences in PD-1 upregulation were even more significant if several rounds of stimulation were performed (FIG. 6B). CAR-Jcam+LCK upregulated PD-1 almost 3-fold higher than CAR-Jcam after three rounds of stimulation. The same upregulation trend was observed for CAR-Jurkat cell samples. The expression of PD-1 in CAR-Jurkat LCK KO was the lowest among the groups, and TCR-Jurkat upregulated PD-1 most strongly.

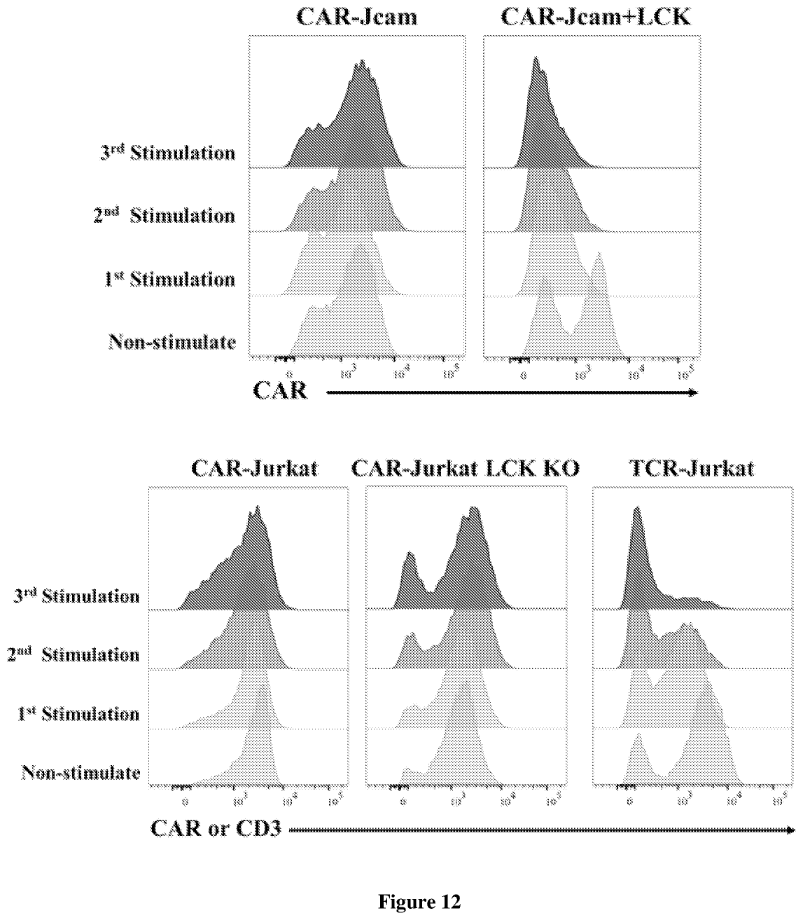

Moreover, CAR or TCR was downregulated after antigen stimulation in the presence of LCK, as shown in a previous study. CAR showed less antigen-dependent receptor downregulation in the LCK-deficient cells, such as Jcam or Jurkat LCK KO (FIG. 6A,D). Compared to the amounts of CAR or TCR prior to stimulation, both were downregulated in CAR-Jurkat and TCR-Jurkat, where TCR was downregulated dramatically. However, the amount of CAR was more stable after antigen stimulation as long as LCK was absent, as seen in CAR-Jcam or CAR-Jurkat LCK KO (FIG. 12). This reduced PD-1 upregulation and CAR downregulation suggested that LCK-deficient CAR-T would be refractory to inhibitory signaling through PD-1 and less prone to “exhaustion” compared with LCK-sufficient CAR-T.

### Example 3

Methods

CD8⁺ T Cell Activation and Culture

Blood samples were collected from volunteers and naïve CD8⁺ T cells isolated by using RosetteSep™ human CD8⁺ T cell enrichment cocktail (Stemcell) and Ficoll (GE Healthcare Life Sciences) gradient centrifugation. Naïve CD8⁺ T cells were then stimulated by anti-CD3/CD28 beads (ThermoFisher) in Biotarget medium (Biological Industry) supplemented with 4% of human platelet lysate (Ultra-GRO™-Advanced, AventaCell) with 100 U/ml IL-2 (R&D System) to produce mature cytotoxic CD8⁺ T cells. After 48 hrs activation, the mature CD8⁺ T cells were cultured in medium containing 100 U/ml IL-2, 10 ng/ml IL-15 and 10 ng/ml IL-7 (R&D System). The medium was changed every 2 days, and cells were replated at 10⁶ cells per ml. The T cells were restimulated by feeder cells, peripheral blood mononuclear cells (PBMC) from donors. PBMC were freshly isolated from blood by gradient centrifugation and irradiated at 30 Gy. PBMC and T cells were resuspended in the same medium at a ratio of 2:1. Final concentrations of 100 U/ml IL-2, 10 ng/ml of IL-7, 10 ng/ml of IL-15 were added to the culture. Lectins from Phaseolus vulgaris (Sigma-Aldrich) was added to the culture at a concentration of 1.5 μg/ml. Blood was collected from healthy volunteers under a protocol approved by NUS IRB. Informed consent was obtained from all donors.

CRISPR-Cas9 Genetic Editing

LCK targeted homologous directed repair (HDR) was previously described. The LCK gRNA2, GCCGGGAAAAGTGATTCGAG (SEQ ID NO: 10), was selected and chemically modified. In brief, the full RNA sequence was, 5′-G*C*C*GGGAAAAGUGAUUCGAGGUUUUAGAGCUAGAAAUAGCAAGUUAAAA UAAGGCUAGUCCGUUAUCAACUUGAAAAAGUGGCACCGAGUCGGUGCU*U*U*U-3′ (SEQ ID NO: 43). Asterisk (*) represents 2′-O-methyl 3′ phosphorothioate. The Cas9-NLS protein (New England Biology) was incubated with LCK gRNA2 at a molecular ratio of 1:2 at 37° C. for 30 mins to form ribonucleoprotein (RNP) complexes. The double strand DNA donor was designed so that 1 kb of the homologous arm was flanking the CAR construct on each side. 120 pmol of RNP and 2 μg of dsDNA were electroporated into 1 million activated CD8⁺ T cells by Amaxa 4D electroporation system (Lonza) via program EH115.

Cytotoxicity Assay

40,000 per well of Daudi or Raji cells were seeded onto U bottom 96 well plates, followed by adding 4,000 to 400,000 of CAR-T or LCK-locus CAR-T cells at the effector to target (E:T) ratio of 0.1:1 to 10:1 per well. The cell mixture was incubated for 18 hrs at 37° C., 5% CO₂. The supernatant was then collected, and the CytoTox 96® Non-Radioactive Cytotoxicity Assay (Promega) was used to detect the release of LDH from dead cells. The cell pellet was suspended and stained with antibody conjugates to detect the expression of CD62L, PD-1, TIM-3, and LAG-3 after encountering target cells.

Mouse Model and In Vivo Analysis

6-8 week-old NOD/MrkBomTac-Prkd^{scid} female mice (Taconic) were used under a protocol approved by the NUS Institutional Animal Care and Use Committee. CAR-T cells are restimulated and expanded at day −3 by feeder cells. Raji cells were administered through tail vein injection at 4 million per mouse. Raji cells produce very even tumor burdens and no mice were excluded before treatment. 5×10⁶, 2.5×10⁶, or 1×10⁶ of Expanded CAR-T cells were administered through the tail vein at day 4 after Raji cells administration. The mice were constantly monitored and euthanized when paralysis was observed. For CAR-T cell phenotyping after in vivo administration, bone marrow of each mouse was extracted at day 11 and day 18. The memory and exhaustion surface markers were detected and analyzed by FACS.

Flow Cytometry and Cell Sorting

Flow cytometry experiments were conducted on BD LSR Fortessa X-20 (Becton Dickinson). Cell sorting was conducted on either Mo-Flo XDP (Beckman Coulter, Inc.) or SY3200 (Sony Biotechnology Inc.) by Flow Cytometry Laboratory, Immunology Programme, National University of Singapore. Data analysis was performed on FlowJo.

Statistical Information and Data Analysis

Two-tailed Student t-test or two-way ANOVA analysis was performed for column data or curve data respectively by using GraphPad Prism 7. The data meet the assumptions of the tests. Variance is similar between the compared groups.

Results

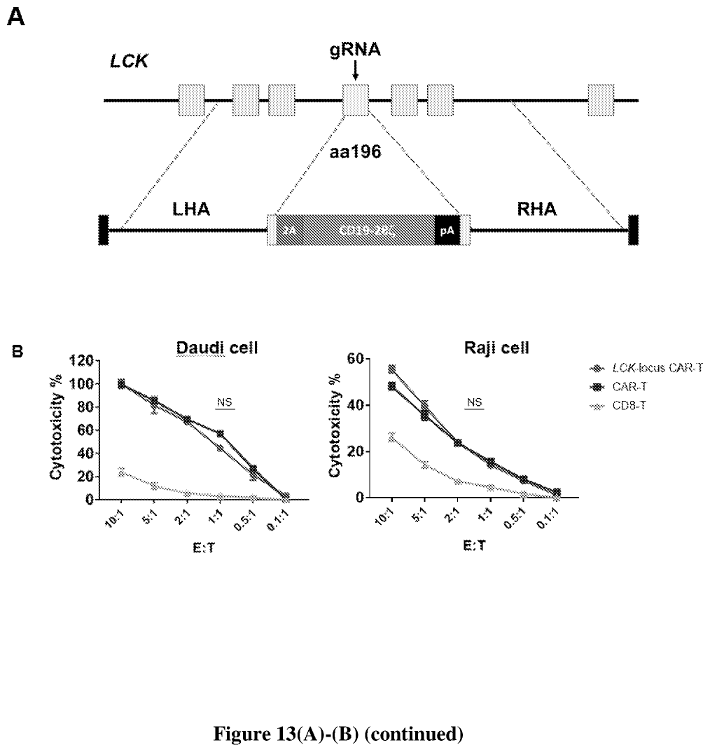

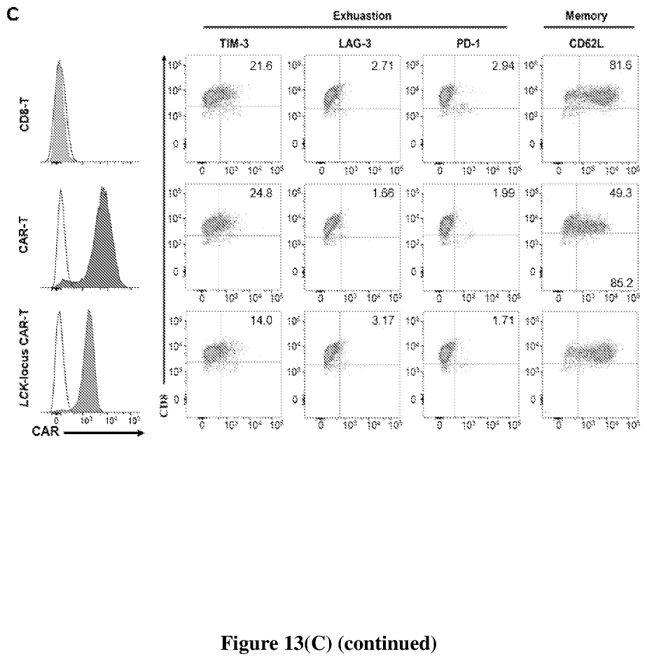

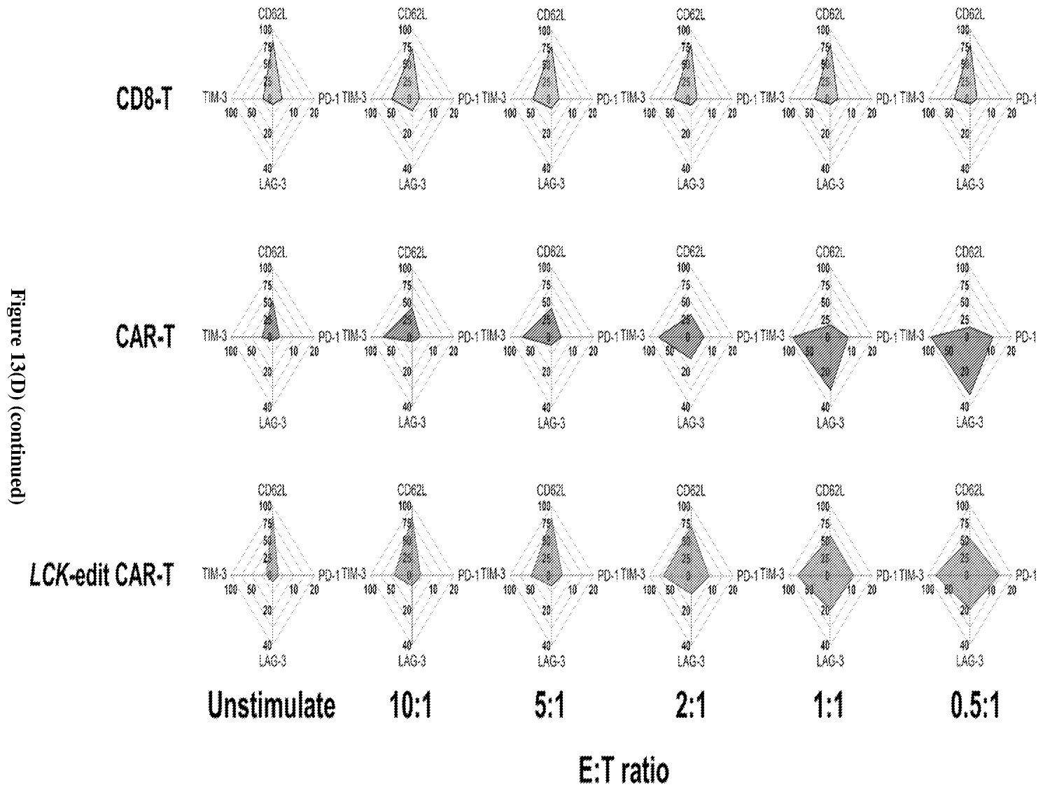

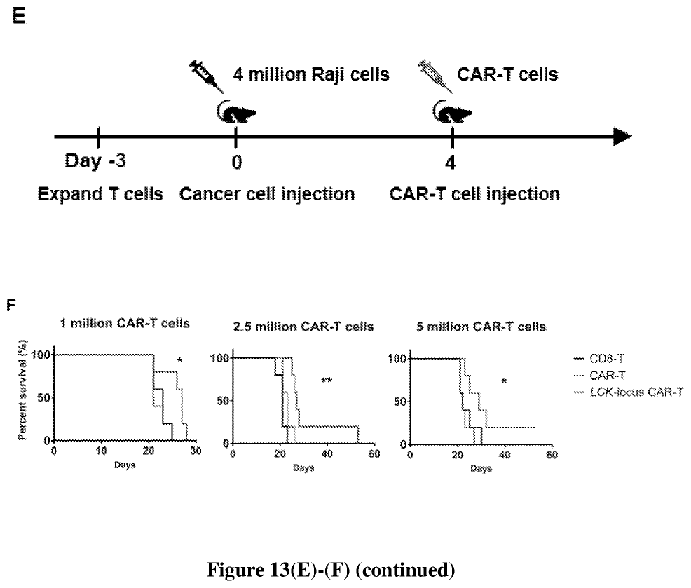

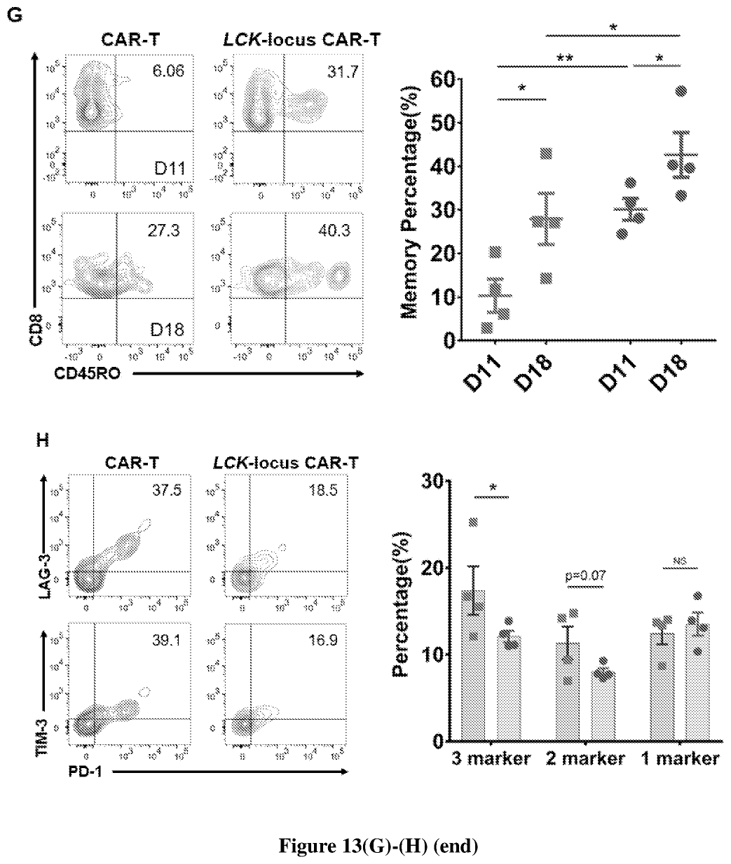

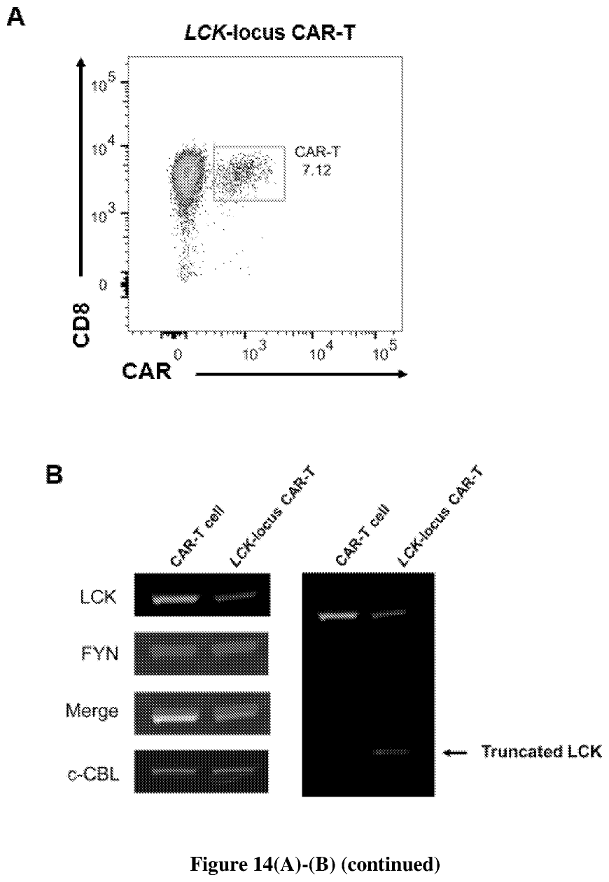

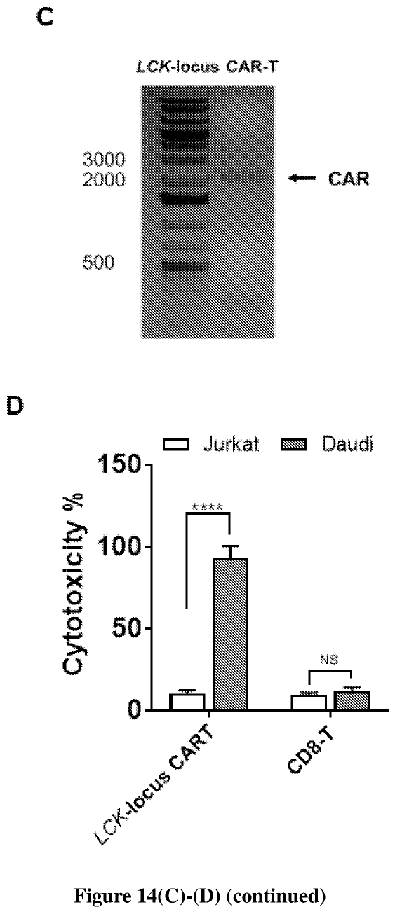

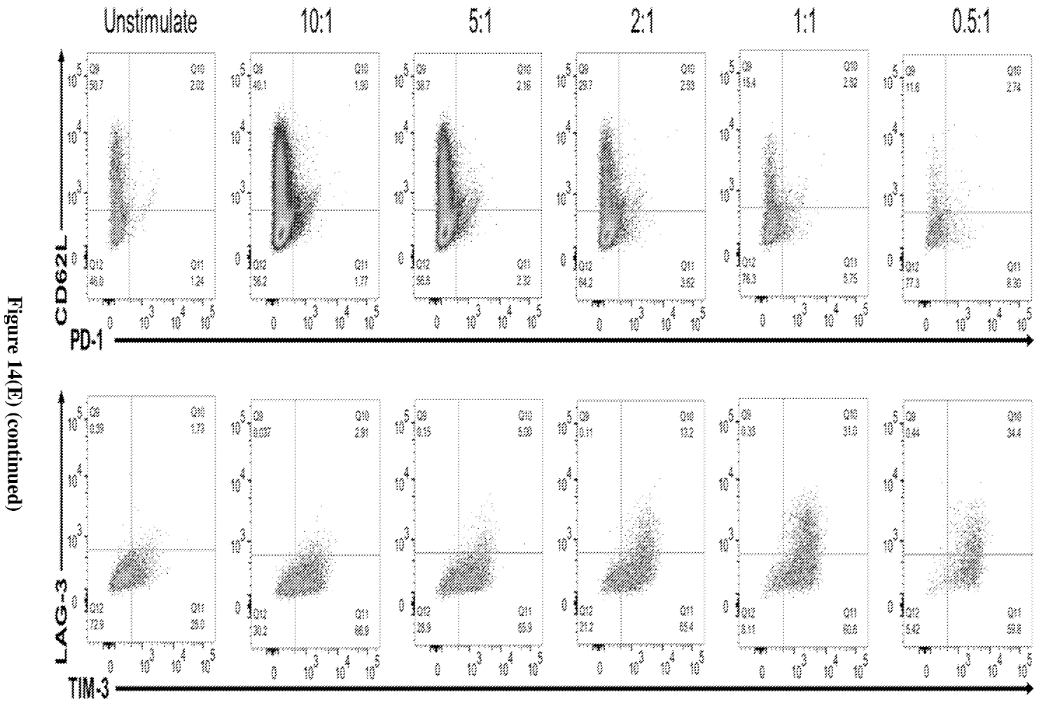

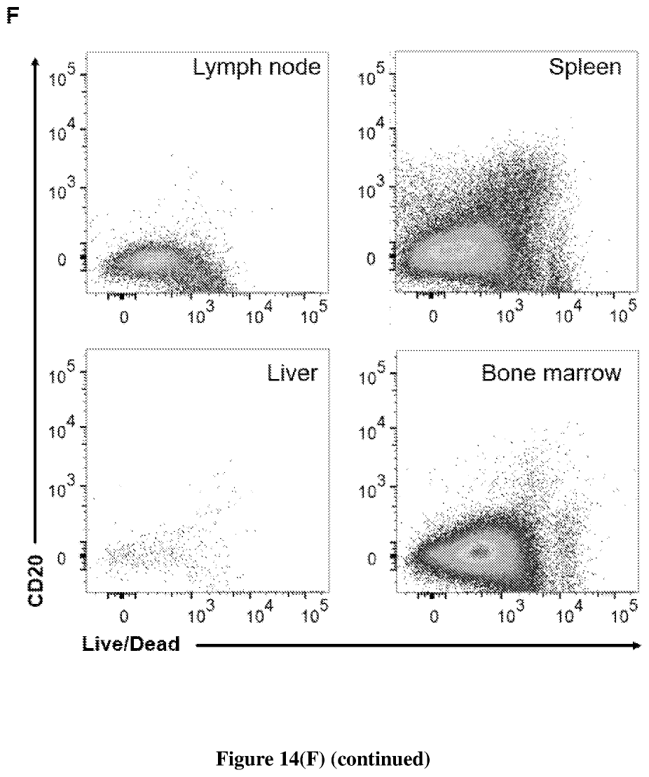

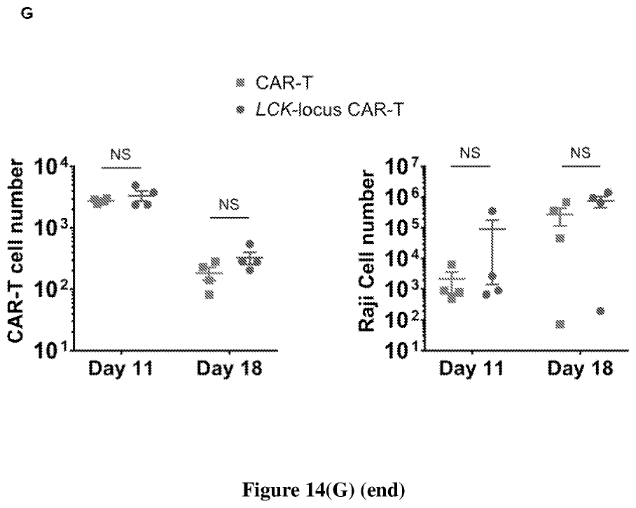

The inventors next sought to recapitulate the findings from Jurkat cells in primary human CD8⁺ T cells. To this end, the CRISPR/Cas9 system was utilized to perform a homologous direct repair (HDR), where the CD28-CAR construct was directed to the LCK locus and inserted at the location of 196^{th} amino acid of LCK (FIG. 13A). A distinct population of CAR⁺ CD8⁺ T cells was detected after HDR (FIG. 14A). The CAR⁺ CD8⁺ T cells were then sorted to measure the LCK protein expression. As shown in FIG. 14B, the LCK expression was dramatically reduced compared with conventional CAR-T cells. A P2A cleavable sequence was added at the N-terminal of CD28-CAR, thus a truncated LCK was also observed (FIG. 14B). This truncated LCK is a dysfunctional variant, given that amino acid 196 is located at the end of LCK's SH2 domain, and is not part of the catalytic domain. Genotyping at the inserted site also confirmed that the CAR construct was inserted into the LCK locus (FIG. 14C). Two CD19-expressing cell lines, Daudi and Raji, were used to test T cell cytotoxicity. Both the conventional CAR-T and LCK-locus CAR-T had comparable cytotoxicity, but they also showed lower cytotoxicity to Raji cells than to Daudi cells. This low cytotoxicity to Raji cells may be caused by a resistant mechanism of Raji cells to T cell killing (FIG. 13B). The control CD8⁺ T cells also showed a certain degree of cytotoxicity against these cancer cells at a high E:T ratio. The specificity of LCK-locus CAR-T cells was retained, as shown in FIG. 14D, as they could only kill CD19-expressing Daudi cells rather than CD19-negative Jurkat cells. Differences between conventional CAR-T and LCK-locus CAR-T were tested by immunotyping of exhaustion molecules, PD-1, TIM-3, LAG-3, and the memory marker CD62L. At resting state, conventional CAR-T cells had higher TIM-3 expression (25%) and lower CD62L expression (49%) than those of LCK-locus CAR-T cells, where TIM-3 expression was 14% and CD62L expression is 85%, respectively (FIG. 13C). After encountering target cells at different E:T ratios, the expression of exhaustion and memory molecules varied (FIG. 14E). At low E:T ratios, higher expression of exhaustion molecules and lower CD62L expression were observed. When radar charts were used to summarize surface marker expression and to compare within different groups, the differences between these two CAR-T cells became more apparent (FIG. 13D). The conventional CAR-T cells were prone to express more exhaustion molecules and reduce the expression of CD62L as E:T decreased. However, LCK-locus CAR-T cells appeared more persistent and were not as prone as conventional CAR-T cells to upregulate exhaustion markers, where expression of PD-1, TIM-3, and LAG-3 was high but CD62L expression was low.

The more persistent phenotype of LCK-locus CAR-T cells, i.e. more memory and less exhausted phenotype, suggested that they would show better in vivo anti-cancer efficacy than conventional CAR-T cells. Conventional CAR-T cells and LCK-locus CAR-T cells were then challenged to tackle Raji cells in a mouse model, where Raji are more resistant to CAR-T cytotoxicity than Daudi cells in vitro (FIG. 13B). This also suggested that they might be more resistant in vivo. A NOD/SCID immune-compromised mouse strain was used, and CAR-T cells were administered i.v. at different doses, followed 4 days later by Raji cells (FIG. 13E). Consistent with our expectation, conventional CAR-T cells did not show significant improvement in efficacy in vivo compared to CD8⁺ T cells. However, LCK-locus CAR-T cells showed significantly enhanced in vivo performance at all three doses from 1 million to 5 million cells (FIG. 13F). At the end of the experiment, one mouse in the group treated with 5 million LCK-locus CAR-T cells was still alive. No cancer cells were discovered within multiple organs extracted from this mouse (FIG. 14F). The Raji and T cells were further analyzed from the bone marrow of the mice at day 11 and day 18 after Raji cells injection to identify their numbers, and the T cells' memory and exhaustion state. The number of T cells and Raji cells from the groups of conventional CAR-T cells and LCK-locus CAR-T cells at day 11 and 18 did not show significant differences (FIG. 14G). However, LCK-locus CAR-T cells showed a significantly higher expression of memory marker CD45RO at both day 11 and 18 compared to conventional CAR-T cells, as seen in FIG. 13G. In addition, the conventional CAR-T cells upregulated the exhaustion markers PD-1, TIM-3 and LAG3, more than LCK-locus CAR-T cells. This was particularly noticeable for the cells co-expressing all three of these exhaustion markers (FIG. 13H). The more memory and less exhausted phenotype of LCK-locus CAR-T cells compared to conventional CAR-T cells, therefore, explained their enhanced in vivo efficacy.

1-32. (canceled)

33. An immune cell expressing a chimeric antigen receptor (CAR),
wherein the CAR comprises an intracellular signaling domain that is functional in the absence of lymphocyte-specific protein tyrosine kinase (LCK), and
wherein expression of LCK is disrupted in the immune cell.

34. The immune cell of claim 33, wherein the intracellular signaling domain comprises a signaling domain of a CD28 protein that is functional in the absence of LCK.

35. The immune cell of claim 33, wherein the immune cell is a T cell or an NK cell.

36. The immune cell of claim 33, wherein the expression of LCK is disrupted using CRISPR editing, Meganuclease editing, TALEN editing or Zinc Finger editing.

37. The immune cell of claim 33, wherein the expression of LCK is disrupted using a nucleic acid that inhibits gene expression of LCK.

38. The immune cell of claim 37, wherein the nucleic acid molecule that inhibits gene expression of LCK is selected from the group consisting of an antisense RNA, antagomir RNA, siRNA, and shRNA.

39. The immune cell of claim 37, wherein the immune cell comprises a vector comprising the nucleic acid that inhibits gene expression of LCK.

40. The immune cell of claim 33, wherein the immune cell comprises a vector comprising a nucleic acid encoding the CAR.

41. The immune cell of claim 33, wherein the immune cell comprises a vector comprising a nucleic acid encoding the CAR and a nucleic acid that inhibits gene expression of LCK.

42. The immune cell of claim 33, wherein the CAR comprises an extracellular antigen-binding domain.

43. The immune cell of claim 33, wherein the intracellular signaling domain further comprises a primary-signaling domain comprising a functional signaling domain of a protein selected from CD3 zeta, CD3 gamma, CD3 delta, CD3 epsilon, FcR gamma, Fc epsilon RI beta, CD79a, CD79b, Fcgamma RIIa, DAP10, or DAP12.

44. The immune cell of claim 33, wherein the intracellular signaling domain further comprises a costimulatory domain comprising a functional signaling domain of a protein selected from the group consisting of DAP10, CD28, CARD11, SLAMF1, LCK1, LCK3, LAT, OX40, CD27, CDS, ICAM-1, LFA-1 (CD11a/CD18), ICOS (CD278), and 4-1BB (CD137).

45. The immune cell of claim 33, wherein the immune cell has reduced PD-1 expression as compared to a cell that has not been modified such that the expression of LCK is inhibited.

46. An immune cell expressing a CAR, wherein the immune cell has been modified such that expression of the LCK gene has been disrupted.

47. A method of manufacturing the immune cell of claim 33, the method comprising disrupting expression of LCK.

48. The method of claim 47, wherein the expression of LCK is disrupted using a nucleic acid that inhibits gene expression of LCK.

49. The method of claim 47, wherein the expression of LCK is disrupted using a CRISPR editing, Meganuclease editing, TALEN editing or Zinc finger editing.

50. The method of claim 47, further comprising introducing a nucleic acid encoding a CAR into the immune cell.
